SEQUENCE LISTING (37 sequences)

SEQ ID NO: 1 - DNA, 595 bp, Hepatitis C virus
gcacgaatcctaaacctcaaagaaaaaccaaacgtaacaccaaccgtcgcccacaggacgtcaagttcccgggtggcggtcagatcgttggtggagtttacttgttgccgcgcaggggccctagattgggtgtgcgcgcgacgaggaagacttccgagcggtcgcaacctcgaggtagacgtcagcctatccccaaggcacgtcggcccgagggcaggacctgggctcagcccgggtacccttggcccctctatggcaatgagggttgcgggtgggcgggatggctcctgtctccccgtggctctcggcctagctggggccccacagacccccggcgtaggtcgcgcaatttgggtaaggtcatcgatacccttacgtgcggcttcgccgacctcatggggtacataccgctcgtcggcgcccctcttggaggcgctgccagggccctggcgcatggcgtccgggttctggaagacggcgtgaactatgcaacagggaaccttcctggttgctctttctctatcttccttctggccctgctctcttgcctgactgtgcccgcttcagcctaccaagtgcgcaattcctcggggct

SEQ ID NO: 2 - protein, 196 aa, Hepatitis C virus
ARILNLKEKPNVTPTVAHRTSSSRVAVRSLVEFTCCRAGALDWVCARRGRLPSGRNLEVDVSLSPRHVGPRAGPGLSPGTLGPSMAMRVAGGRDGSCLPVALGLAGAPQTPGVGRAIWVRSSIPLRAASPTSWGTYRSSAPLLEAAPGPWRMASGFWKTATMQQGTFLVALSLSSFWPCSLALCPLQPTKCAIPRG

SEQ ID NO: 3 - protein, 13 aa, Hepatitis C virus
  Variant 8: Xaa = Asn or Lys
LNLKEKPXXTPTX

SEQ ID NO: 4 - protein, 13 aa, Hepatitis C virus
  Variant 6: Xaa = Leu or Ser
AAHRTXSSRXXVR

SEQ ID NO: 5 - protein, 14 aa, Hepatitis C virus
LNLKEKPNVTPTAC

SEQ ID NO: 6 - protein, 14 aa, Hepatitis C virus
AAHRTSSSRAVVRC

SEQ ID NO: 7 - protein, 18 aa, Hepatitis C virus
PTDPRRRSRNLGKVIDTC

SEQ ID NO: 8 - protein, 20 aa, Hepatitis C virus
GCATRKTSERSQPRGRRAPI

SEQ ID NO: 9 - protein, 100 aa, Hepatitis C virus
GALDWVCARRGRLPSGRNLEVDVSLSPRHVGPRAGPGLSPGTLGPSMAMRAAGGRDGSCLPVALGLAGAPQTPGVGRAIWVRSSIPLRAASPTSWGTYRS

SEQ ID NO: 10 - protein, 100 aa, Hepatitis C virus
  VARIANT 24: Xaa =Ser or Asn
GAPGWVCARLGRLPSGRNLVEGDXLSPRLAGPRAGPGLSPGTLGPSMAMRAWGGQDGSCPPAALGLVGAPRTPGVGRAIWVRSSIPSHAASPTSWGTFRS

SEQ ID NO: 11 - protein, 90 aa, Hepatitis C virus
GRLPSGRSLVEGASLSPRIAGPRAGPGLSPGTLGPSMAMRVAGGQDGSCPPAALGLLGAPMTPGGGPAIWVRSSIPLRAASPTSWGTSRS

SEQ ID NO: 12 - protein, 161 aa, Hepatitis C virus
MARILNLKEKPNVTPTVAHRTSSSRVAVRSLVEFTCCRAGALDWVCARRGRLPSGRNLEVDVSLSPRHVGPRAGPGLSPGTLGPSMAMRAAGGRDGSCLPVALGLAGAPQTPGVGRAIWVRSSIPLRAASPTSWGTYRSSAPLLEALPGPWRMASGFWKTA

SEQ ID NO: 13 - protein, 13 aa, Hepatitis C virus
LNLKEKPNVTPTA

SEQ ID NO: 14 - protein, 13 aa, Hepatitis C virus
AAHRTSSSRAVVR

SEQ ID NO: 15 - protein, 19 aa, Hepatitis C virus
RAGAPGWVCARLGRLPSGR

SEQ ID NO: 16 - protein, 160 aa, Hepatitis C virus
MARILNLKEKPNVTPTVAHRTSSSRVAVRSLVEFTCCRAGALDWVCARRGRLPSGRNLEVDVSLSPRHVGPRAGPGLSPGTLGPSMAMRAAGGRDGSCLPVALGLAGAPQTPGVGRAIWVRSSIPLRAASPTSWGTYRSSAPLLEALPGPWRMASGFWKT

SEQ ID NO: 17 - protein, 18 aa, Hepatitis C virus
MARILNLKEKPNVTPTVA

SEQ ID NO: 18 - protein, 18 aa, Hepatitis C virus
PNVTPTVAHRTSSSRVAV

SEQ ID NO: 19 - protein, 18 aa, Hepatitis C virus
TSSSRVAVRSLVEFTCCR

SEQ ID NO: 20 - protein, 18 aa, Hepatitis C virus
LVEFTCCRAGALDWVCAR

SEQ ID NO: 21 - protein, 18 aa, Hepatitis C virus
ALDWVCARRGRLPSGRNL

SEQ ID NO: 22 - protein, 18 aa, Hepatitis C virus
RLPSGRNLEVDVSLSPRH

SEQ ID NO: 23 - protein, 17 aa, Hepatitis C virus
DVSLSPRHVGPRAGPGL

SEQ ID NO: 24 - protein, 18 aa, Hepatitis C virus
GPRAGPGLSPGTLGPSMA

SEQ ID NO: 25 - protein, 18 aa, Hepatitis C virus
GTLGPSMAMRAAGGRDGS

SEQ ID NO: 26 - protein, 17 aa, Hepatitis C virus
AAGGRDGSCLPVALGLA

SEQ ID NO: 27 - protein, 18 aa, Hepatitis C virus
LPVALGLAGAPQTPGVGR

SEQ ID NO: 28 - protein, 18 aa, Hepatitis C virus
PQTPGVGRAIWVRSSIPL

SEQ ID NO: 29 - protein, 19 aa, Hepatitis C virus
WVRSSIPLRAASPTSWGTY

SEQ ID NO: 30 - protein, 18 aa, Hepatitis C virus
SPTSWGTYRSSAPLLEAL

SEQ ID NO: 31 - protein, 18 aa, Hepatitis C virus
SAPLLEALPGPWRMASGF

SEQ ID NO: 32 - protein, 18 aa, Hepatitis C virus
RMASGFPWRMASGFWKTA

SEQ ID NO: 33 - protein, 70 aa, Hepatitis C virus
MARILNLKEKPNVTPTVAHRTSSSRVAVRSLVEFTCCRAGALDWVCARRGRLPSGRNLEVDVSLSPRHVG

SEQ ID NO: 34 - protein, 70 aa, Hepatitis C virus
CARRGRLPSGRNLEVDVSLSPRHVGPRAGPGLSPGTLGPSMAMRAAGGRDGSCLPVALGLAGAPQTPGVG

SEQ ID NO: 35 - protein, 70 aa, Hepatitis C virus
GGRDGSCLPVALGLAGAPQTPGVGRAIWVRSSIPLRAASPTSWGTYRSSAPLLEALPGPWRMASGFWKTA

SEQ ID NO: 36 - DNA, 495 bp, Hepatitis C virus
gccaccatggccagaatcctgaacctgaagaagaaaaccaacgtgacccccaccgtggcccacaggaccagcagcagcagagtggccgtgaggagcctggtggagttcacctgctgcagggccggagccctggactgggtgtgcgccaggagggagaggctgcccagcggcaggaacctggaagtggacgtgtctctgagccccagactggtgggccccagagccggacccggcctgtcccctggcaccctgggccctagcatggctatgagggccgcaggcggaagggacggcagctgtctgcccgtggcactgggcctggctggcgcccctcagacacctggcgtgggcagagccatctgggtgagaagcagcatccccctgagagccgcctcccctacctcttggggcacctacaggagcagcgccccactgctggaagccctgcctggcccttggaggatggcctctggcttctggaaaaccgcctgatga

SEQ ID NO: 37 - protein, 161 aa, Hepatitis C virus
MARILNLKKKTNVTPTVAHRTSSSRVAVRSLVEFTCCRAGALDWVCARRERLPSGRNLEVDVSLSPRLVGPRAGPGLSPGTLGPSMAMRAAGGRDGSCLPVALGLAGAPQTPGVGRAIWVRSSIPLRAASPTSWGTYRSSAPLLEALPGPWRMASGFWKTA

(19) United States
(12) Patent Application Publication
(10) Pub. No.: US 2005/0202036 A1
(43) Pub. Date: Sep. 15, 2005

(54) Alternate reading frame polypeptides drived from hepatitis C and methods of their use

(76) Inventors: Andrea D. Branch, New York, NY (US); Jose L. Walewski, Eastchester, NY (US); Decherd D. Stump, New York, NY (US)

Correspondence Address: LAHIVE & COCKFIELD, LLP., 28 STATE STREET, BOSTON, MA, 02109, US

(21) Appl. No.: 10/897,680

(22) Filed: Jul. 22, 2004

Related U.S. Application Data
(60) Provisional application No. 60/489,544, filed on Jul. 22, 2003.

(51) Int. Cl. C07K 14/02; A61K 39/29

(52) U.S. Cl. 424/189.1; 530/350

(57) ABSTRACT

Novel hepatitis C virus (HCV) polypeptides are provided which are not encoded by the standard HCV open reading frame. These alternate reading frame polypeptides are useful, inter alia, in vaccine compositions, in diagnosing HCV infection, and as therapeutic targets.

### RELATED APPLICATIONS

This application claims the benefit of U.S. Ser. No. 60/489,544, “Alternate Reading Frame Polypeptides Derived from Hepatitis C and Methods of Their Use,” filed Jul. 22, 2003. This application is also related to U.S. Ser. No. 60/088,670, “Novel Hepatitis C Polypeptides” filed on Jun. 9, 1998; U.S. Ser. No. 60/089,138, “Novel Hepatitis C Polypeptides,” filed on Jun. 11, 1998; PCT/US99/12929, “Novel Hepatitis C Virus Peptides and Uses Thereof,” filed on Jun. 9, 1999; U.S. Ser. No. 09/719,277, “Novel Hepatitis C Virus Peptides and Uses Thereof,” filed on Apr. 13, 2001; and U.S. Ser. No. 10/601,020, “Novel Hepatitis C Virus Peptides and Uses Thereof,” filed Jun. 20, 2003. The contents of each of these applications is incorporated by reference herein.

### GOVERNMENT FUNDING

This work was funded, in part, by NIH grants DK52071, DK50795. The government may, therefore, have certain rights to this invention.

### BACKGROUND OF THE INVENTION

Hepatitis C virus (HCV) is closely related to both the pestivirus and flavivirus genera in the Flaviviridae family. HCV is a single stranded RNA virus; the viral genome is approximately 9.5 kb. HCV RNA is positive sense and has a unique open reading frame which encodes a single polyprotein (Clarke. 1997. J. Gen. Virol. 78:2397). The polyprotein is proteolyticly processed to yield the mature viral proteins which include: nucleocapsid, envelope 1, envelope 2, metalloprotease, serine protease, RNA helicase, cofactor, and RNA polymerase. The HCV genome has a high degree of genetic variability. Interindividual HCV sequence variability has led to the classification of at least six genotypes.

HCV is a major human pathogen. The virus was found to be the cause of most cases of hepatitis which could not be ascribed to hepatitis A, hepatitis B, or hepatitis delta virus (Clarke, supra). Over fifty percent of patients with hepatitis C virus (HCV) become chronic carriers of the virus; there may be as many as 500 million chronic carriers worldwide (Dhillon and Ducheiko. 1995. Histopathology 26: 297). Persistent infection with the virus causes chronic hepatitis and may ultimately lead to cirrhosis and/or cancer (Kuo et al. 1989. Science 244:362). Current therapies for HCV are ineffective, consequently there is a need for new approaches to treat and diagnose HCV infection.

### SUMMARY

The present invention is an important advance in the battle against hepatitis C. The polypeptides of the invention comprise naturally occurring HCV polypeptides, or consensus sequences derived therefrom, which are not encoded by the standard, polyprotein HCV reading frame. These alternate reading frame polypeptides elicit an immune response in patients infected with HCV and, thus, are produced during HCV infection. Accordingly, the invention provides polypeptides comprising HCV alternate reading frame amino acid sequences which are not derived from the HCV polyprotein (e.g., purified or recombinant naturally occurring alternate reading frame polypeptides and consensus polypeptides based on these sequences) and methods of their use.

In one aspect, the invention pertains to an isolated, synthetic, or recombinant polypeptide of at least about 100 amino acids in length, wherein the polypeptide is immunoreactive with antisera that specifically detects an HCV alternate reading frame polypeptide relative to an HCV standard reading frame polypeptide.

In one embodiment, the polypeptide comprises an amino acid sequence encoded by a reading frame +1 to the main HCV open reading frame.

In one embodiment, the polypeptide comprises the sequence:

GALDWVCARRGRLPSGRNLEVDVSLSPRHVGPRAGPGLSPGTLGPSMAM
RAAGGRDGSCLPVALGLAGAPQTPGVGRAIWVRSSIPLRAASPTSWGTY
RS.

In one embodiment, the polypeptide comprises the sequence:

GAPGWVCARLGRLPSGRNLVEGD(S/N)LSPRLAGPRAGPGLSPGTLGPS
MAMRAWGGQDGSCPPAALGLVGAPRTPGVGRAIWVRSSIPSHAASPTSWG
TFRS.

In one embodiment, the polypeptide comprises the sequence:

GRLPSGRSLVEGASLSPRIAGPRAGPGLSPGTLGPSMAMRVAGGQDGSCP
PAALGLLGAPMTPGGGPAIWVRSSIPLRAASPTSWGTSRS

In one embodiment, the polypeptide comprises an amino acid sequence that varies from the consensus amino acid sequence shown in FIG. 9 by substitution of between about one and about twenty amino acids.

In one embodiment, the polypeptide further comprises a non-HCV polypeptide or other material.

In one embodiment, the non-HCV polypeptide or other material increases an immune response to the polypeptide.

In one embodiment, the polypeptide further comprises an amino acid sequence encoded by the HCV standard reading frame.

In another aspect the invention pertains to polypeptide comprising at least eight consecutive amino acids of the amino acid sequence:

MARILNLKEKPNVTPTVAHRTSSSRVAVRSLVEFTCCRAGALDWVCARRG
RLPSGRNLEVDVSLSPRHVGPRAGPGLSPGTLGPSMAMRAAGGRDGSCLP
VALGLAGAPQTPGVGRAIWVRSSIPLRAASPTSWGTYRSSAPLLEALPGP
WRMASGFWKTA

In one embodiment, the invention pertains to a composition comprising the polypeptide of claim 1 and a pharmaceutically acceptable carrier.

In another embodiment, the invention pertains to a method of preventing HCV infection comprising administering the composition of claim 11 to a subject.

In another aspect, the invention pertains to a method of preventing chronic HCV infection comprising administering at least one isolated, synthetic, or recombinant polypeptide to a subject, wherein the polypeptide comprises an immunogenic portion of an HCV alternate reading frame polypeptide.

In one embodiment, the invention pertains to a method of preventing chronic HCV infection comprising administering the polypeptide of claim 11 to a subject.

In one embodiment, the invention pertains to a method of increasing clearance of HCV in a subject during an ongoing infection comprising administering at least one isolated, synthetic, or recombinant HCV polypeptide to the subject, wherein the polypeptide comprises an immunogenic portion of an HCV alternate reading frame polypeptide.

In another embodiment, the invention pertains to a method of increasing clearance of HCV comprising administering the polypeptide of claim 11 to a subject.

In another aspect, the invention pertains to a method of predicting the response of a subject to a therapy for HCV comprising detecting the presence or absence of a polypeptide in the body fluid or cells of a subject, wherein the polypeptide is immunoreactive with antisera that specifically detects an HCV alternate reading frame polypeptide relative to an HCV standard reading frame polypeptide, or the presence or absence of antibodies which bind to the polypeptide, wherein the presence of the polypeptide or antibodies which bind to the polypeptide is predictive of a favorable response to therapy for HCV.

In one embodiment, the therapy comprises administration of IFN.

In one aspect, the invention pertains to a method of predicting the propensity of a patient infected with an HCV virus to clear HCV spontaneously, to clear HCV in response to IFN, to progress to chronic infection, to fail IFN treatment, to develop chronic HCV disease, cirrhosis, or liver cancer comprising detecting sequence changes in the amino acid sequence of alternate reading frame polypeptides made by the virus or the RNA molecules encoding them.

In one embodiment, a polypeptide is conjugated to a moiety that facilitates detection or attachment to a matrix.

In one embodiment, the polypeptide is immobilized on a matrix.

In one aspect, the invention pertains to a method of diagnosing acute HCV infection comprising detecting the presence or absence of a polypeptide comprising an amino acid sequence encoded by an HCV alternate reading frame or detecting the presence or absence of antibodies which bind to a polypeptide comprising an amino acid sequence encoded by an HCV alternate reading frame in the body fluid or cells of a subject, wherein the presence of the polypeptide or antibodies which bind to the polypeptide is indicative of acute infection with HCV.

In another aspect, the invention pertains to a method of diagnosing acute HCV infection, comprising detecting the presence or absence of antibodies in the body fluid of a subject, wherein the antibodies react with a polypeptide comprising an amino acid sequence encoded by an HCV alternate reading frame or a consensus polypeptide comprising a consensus sequence based on HCV alternate reading frame proteins encoded by more than one viral genotype, and wherein the presence of the antibodies is indicative of an acute infection with HCV.

In one aspect, the invention pertains to a method of diagnosing acute Hepatitis C virus (HCV) infection, comprising detecting the presence or absence of a polypeptide in the body fluid or cells of a subject, wherein the polypeptide comprises an amino acid sequence encoded by an HCV alternate reading frame, and wherein the presence of the polypeptide is indicative of acute infection with HCV.

In one embodiment, the invention pertains to a method of diagnosing liver cancer or advanced cirrhosis comprising detecting the presence or absence of a polypeptide comprising an amino acid sequence encoded by an HCV alternate reading frame or detecting the presence or absence of antibodies which bind to a polypeptide comprising an amino acid sequence encoded by an HCV alternate reading frame in the body fluid or cells of a subject, wherein the presence of the polypeptide or antibodies which bind to the polypeptide is indicative of liver cancer or advanced cirrhosis.

In one aspect, the invention pertains to a method of diagnosing liver cancer or advanced cirrhosis, comprising detecting the presence or absence of antibodies in the body fluid of a subject, wherein the antibodies react with a polypeptide comprising an amino acid sequence encoded by an HCV alternate reading frame or a consensus polypeptide comprising a consensus sequence based on HCV alternate reading frame proteins encoded by more than one viral genotype, and wherein the presence of the antibodies is indicative of liver cancer or advanced cirrhosis.

In one embodiment, the invention pertains to a method of diagnosing liver cancer or advanced cirrhosis, comprising detecting the presence or absence of a polypeptide in the body fluid or cells of a subject, wherein the polypeptide comprises an amino acid sequence encoded by an HCV alternate reading frame, and wherein the presence of the polypeptide is indicative of liver cancer or advanced cirrhosis.

In one embodiment, the presence or absence of the polypeptide is detected by contacting a test sample comprising body fluid or cells of a subject with an antibody under conditions which allow the binding of the polypeptide to the antibody and wherein the binding of the antibody to the polypeptide in the sample indicates the presence of an HCV alternate reading frame polypeptide.

In one embodiment, the presence or absence of antibodies is detected by contacting a test sample comprising body fluid of a subject with the polypeptide under conditions which allow the binding of the polypeptide to the antibody and wherein the binding of the polypeptide to the antibody in the sample indicates the presence of an HCV alternate reading frame polypeptide.

In one embodiment, the polypeptide comprises an amino acid sequence that differs in sequence from a naturally occurring HCV alternate reading frame polypeptide by at least one amino acid residue.

In one embodiment, the entire polypeptide corresponds to a polypeptide encoded by a reading frame +1 or +2 to the standard hepatitis C reading frame.

In one embodiment, the polypeptide comprises an amino acid sequence at least about 80% identical to the consensus amino acid sequence shown in FIG. 9 using FASTA alignment.

In one embodiment, the polypeptide comprises an amino acid sequence at least about 90% identical to the consensus amino acid sequence shown in FIG. 9 using FASTA alignment.

In one embodiment, the polypeptide comprises an amino acid sequence at least about 95% identical to the consensus amino acid sequence shown in FIG. 9 using FASTA alignment.

In one embodiment, the antibody is polyclonal.

In one embodiment, the antibody is monoclonal.

In one embodiment, the invention pertains to an antibody that recognizes an alternate reading frame polypeptide.

In one aspect, the invention pertains to a kit for detection of HCV or for detecting a particular clinical state comprising an antibody that binds to an alternate reading frame polypeptide.

In another embodiment, the invention pertains to a kit for detection of HCV or for detecting a particular clinical state comprising an alternate reading frame polypeptide.

In another aspect, the invention pertains to a method for determining the presence or absence of antibodies that recognize the polypeptide of claim 1 in a test sample, comprising:
i) adsorbing at least one polypeptide of claim 1 to a matrix;
ii) contacting the test sample with the polypeptide(s) under conditions which allow the binding of antibodies present in the test sample to the polypeptide;
iii) removing the unbound sample from the matrix; and
iv) detecting the presence or absence of antibodies bound to the polypeptide(s) to thereby determine the presence or absence of antibodies that recognize the polypeptide of claim 1 in a test sample.

In another aspect, the invention pertains to a method for identifying a compound which interacts with the polypeptide of claim 1 or the nucleic acid molecule encoding the polypeptide, comprising:
contacting said polypeptide with a compound in a cell-free system under conditions which allow interaction of the compound with the polypeptide or the nucleic acid molecule encoding the polypeptide such that a complex is formed;
separating the compounds which do not form complexes with the polypeptide or the nucleic acid molecule encoding the polypeptide from those which do form complexes with the polypeptide or the nucleic acid molecule encoding the polypeptide; and
isolating and identifying the compounds which form complexes with the polypeptide or the nucleic acid molecule encoding the polypeptide to thereby identify a compound which interacts with the polypeptide of claim 1 or the nucleic acid molecule encoding the polypeptide.

In one embodiment, the invention pertains to a compound identified using a method of the invention.

### BRIEF DESCRIPTION OF THE FIGURES

FIG. 1 shows potential recoding signals in HCV Core/ARF RNA.

FIG. 2 shows exemplary schemes for production of alternate reading frame polypeptides and frameshift back into the standard ORF.

FIG. 3 shows an exemplary alignment of alternate reading frame polypeptide sequences that could be used to make a consensus polypeptide.

FIG. 4 shows an example a log odds substitution matrix that shows, e.g., how representative a reference sequence (e.g. a consensus sequence) is of a population of sequences.

FIG. 5 shows mutations identified in the 5′ Terminal-Stem loop in patients producing alternate reading frame polypeptides.

FIG. 6 shows the expected molecular weight of a consensus polypeptide as determined by Mass spectroscopy

FIG. 7 shows that an alternate reading frame polypeptide reacts with serum from an HCV patient, but not from a control as determined by Western Blotting.

FIG. 8 shows the results of a Western Blot analysis of lysates from HEK293T cells transiently transfected with pARFΔ-hrGFP which expresses the ARFP from a codon optimized sequence.

FIG. 9 shows a consensus amino acid sequence of an alternate reading frame polypeptide for HCV genotype 1a. Amino acid sequences for exemplary clones used in making the consensus sequence are also shown.

FIG. 10 shows a consensus amino acid sequence of an alternate reading frame polypeptide for HCV genotype 1a and a panel of peptides derived from the conensus sequence. Each panel is approximately 18 amino acids in length. The peptides span the entire consensus sequence, starting at the amino terminus with approximately 8 amino acid overlaps.

FIG. 11 shows a panel of peptides, each approximately 70 amino acids in length, derived from the 1a consensus sequence.

### DETAILED DESCRIPTION

The present invention is an important step forward in preventing Hepatitis C (HCV) infection, in treating ongoing infection, and in improving existing diagnostic techniques. The invention is based, in part, on the identification of novel polypeptides encoded by the Hepatitis C viral genome. These novel polypeptides comprise an amino acid sequence which is not encoded by the standard HCV polyprotein reading frame.

Before further description of the invention, certain terms employed in the specification, examples and appended claims are, for convenience, collected here.

I. Definitions

As used herein the term “HCV alternate reading frame polypeptide” includes polypeptides which comprise an amino acid sequence encoded by translation of an HCV nucleic acid molecule in an alternate reading frame, i.e., a reading frame other than the standard open reading frame. Such polypeptides comprise an amino acid sequence which is a naturally-occurring alternate reading frame polypeptide sequence or which is a consensus sequence (based on the amino acid sequence of naturally occurring hepatitis C virus (HCV) alternate reading frame polypeptides).

In one embodiment, such polypeptides comprise an immunogenic portion of an HCV alternate reading frame amino acid sequence, i.e., a portion of an amino acid sequence encoded by an HCV alternate reading frame sufficient to elicit an immune response, e.g., about 8 amino acids in length. In one embodiment, a polypeptide elicits an T cell response. In another embodiment, a polypeptide elicits a B cell response. In another embodiment, an HCV alternate reading frame polypeptide comprises an immunoreactive portion of an amino acid sequence encoded by an HCV alternate reading frame, i.e., an amino acid sequence which is bound by at least one antibody which specifically binds to an HCV alternate reading frame protein relative to a standard HCV open reading frame protein.

As used herein, the language “the standard hepatitis C virus open reading frame” is the open reading frame (ORF) of the viral RNA which encodes the well-known HCV polyprotein. The standard ORF represents the largest ORF in the viral genome. In the infectious clone (GenBank accession number AF011751) the standard ORF uses nucleotide 342 as the first nucleotide of a codon and continues until nucleotide 9377. In different HCV isolates, the nucleotide which is a first nucleotide of a codon of the standard ORF may be at a slightly different position. The nucleotide which is a first nucleotide of a codon for any isolate can be easily be obtained to yield the standard HCV ORF. For example, in the case of known isolates GenBank (or another database containing the nucleotide sequence information for the isolate) can be accessed and the coding sequence (CDS) information can be obtained. Alternatively, to determine the standard ORF of a known or a new isolate, the nucleic acid sequence of the known or new isolate can be aligned with a known sequence to give the highest homology (e.g., using a program such as BLAST). An exemplary BLAST search can be done, e.g., using the sequence found in GenBank accession number AF011751, as the query sequence. In this search, nucleotides 342-940 of AF011751 were used to search the non-redundant sequence database. The ORF of other HCV isolates which corresponds to the standard HCV ORF of AF011751 (in which the initiation codon is at position 342, which is read as position 1 of the query sequence) can be read from the BLAST alignment. For example, the corresponding first nucleotide of a codon for GenBank accession no. HPCCGAA is 342. Another way to find the standard ORF would be to use a program, such as Edit Seq. (DNASTAR) which is designed to identify ORFS using the AUG aligned with position 342 of AF011751 as the start codon.

As used herein, the language “isolated or recombinant polypeptide” includes a polypeptide which is substantially free of cellular material or culture medium when produced by recombinant DNA techniques, or chemical precursors or other chemicals when chemically synthesized.

As used herein, the term “polypeptide or fragment thereof” includes full-length polypeptide molecules (from the first amino acid of translation initiation to the last amino acid prior to translation termination) and peptide portions of such molecules. Preferably the polypeptides comprising an amino acid sequence encoded by an HCV alternate reading frame or fragments thereof are at least about 8 amino acids to at least about 200 amino acids in length. In preferred embodiments, the polypeptides comprising an amino acid sequence encoded by an HCV alternate reading frame of the invention comprise a consensus amino acid sequence which incorporates amino acid residues that are conserved among different HCV isolates. Such a consensus sequence can readily be determined using an alignment such as that provided in Table 1.

As used herein the term “non-HCV polypeptide” includes polypeptides that are not HCV polypeptides.

As used herein, the term amino acid “consensus sequence” includes an amino acid sequence derived from a comparison of naturally occurring polypeptide sequences encoded by more than one viral variant, subgenotype, and/or genotype. In one embodiment, the consensus sequence includes an amino acid residue at each position of the polypeptide that represents the amino acid residue at that position in the majority of the polypeptides compared. In a preferred embodiment, an HCV alternate reading frame consensus sequence is a genotype 1a consensus sequence.

In other embodiments, the polypeptides comprising an amino acid sequence encoded by an HCV alternate reading frame comprise a portion of a HCV alternate reading frame amino acid sequence which is distal (carboxy terminal) to a stop codon in the +1 reading frame (relative to the main ORF) that includes the “UG” of the “AUG” that is the initiator codon of the main ORF. In a more preferred embodiment, the polypeptides or fragments thereof cause an immune response in a subject.

As used herein, the language “nucleic acid molecule” is intended to include DNA molecules (e.g., cDNA or genomic DNA) and RNA molecules (e.g., genomic viral RNA or mRNA). The nucleic acid molecule may be single-stranded or double-stranded.

The term “percent (%) identity” as used in the context of nucleotide and amino acid sequences (e.g., when one amino acid sequence is said to be X % identical to another amino acid sequence) refers to the percentage of identical residues shared between the two sequences, when optimally aligned. To determine the percent identity of two nucleotide or amino acid sequences, the sequences are aligned for optimal comparison purposes (e.g., gaps may be introduced in one sequence for optimal alignment with the other sequence). The residues at corresponding positions are then compared and when a position in one sequence is occupied by the same residue as the corresponding position in the other sequence, then the molecules are identical at that position. The percent identity between two sequences, therefore, is a function of the number of identical positions shared by two sequences (i.e., % identity=# of identical positions/total # of positions×100).

Computer algorithms known in the art can be used to optimally align and compare two nucleotide or amino acid sequences to define the percent identity between the two sequences. A preferred, non-limiting example of a mathematical algorithm utilized for the comparison of two sequences is the algorithm of Karlin and Altschul (1990) Proc. Natl. Acad. Sci. USA 87:2264-68, modified as in Karlin and Altschul (1993) Proc. Natl. Acad. Sci. USA 90:5873-77. Such an algorithm is incorporated into the NBLAST and XBLAST programs of Altschul, et al. (1990) J. Mol. Biol. 215:403-10. To obtain gapped alignments for comparison purposes, Gapped BLAST can be utilized as described in Altschul et al., (1997) Nucleic Acids Research 25(17):3389-3402. When utilizing BLAST and Gapped BLAST programs, the default parameters of the respective programs (e.g., XBLAST and NBLAST) can be used. See http://www.ncbi.nlm.nih.gov.

Another preferred, non-limiting example of a mathematical algorithm utilized for the comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. If multiple programs are used to compare sequences, the program that provides optimal alignment (i.e., the highest percent identity between the two sequences) is used for comparison purposes.

As used herein, the language “+1 or +2 relative to the standard hepatitis C virus open reading frame” includes reading frames in which a first nucleotide of a codon is shifted +1 nucleotide relative to the standard ORF or +2 nucleotide relative to the standard ORF. The reading frames encoding the novel polypeptides do not necessarily contain an in-frame start codon. It will be understood that the terms “+1” and “+2” include other possible starting points that ultimately are translated in the +1 or +2 reading frame (e.g., −1, −2, +4, +5, etc.)

As used herein, the language “the reading frame of SEQ ID NO:1” means that the first three nucleotides of the sequence shown in SEQ ID NO:1 are the first second and third nucleotides of a codon for translation into an amino acid of a polypeptide. The reading frame of SEQ ID NO:1 is +1 relative to the standard HCV ORF. The language “a reading frame corresponding to the reading frame of SEQ ID NO:1” means that when a sequence from an HCV isolate other than the AF011751 isolate shown in SEQ ID NO:1 is aligned with the sequence of SEQ ID NO:1 to give the highest homology, e.g., using the BLAST program, it is then read in the same reading frame as SEQ ID NO:1 to give the reading frame corresponding the reading frame of SEQ ID NO:1. The nucleotide position of a first nucleotide of a codon of an HCV isolate which corresponds to that of SEQ ID NO:1 may vary from isolate to isolate. For example, for GenBank accession number AF009606 a first nucleotide of a codon in a reading frame which corresponds to the reading frame of SEQ ID NO:1 is nucleotide 346.

As used herein, the term “hybridizes under high stringency” is intended to describe conditions for hybridization and washing under which nucleotide sequences at least 70% homologous to each other typically remain hybridized to each other. Preferably, the conditions are such that sequences at least 75%, 85%, or 95% identical to each other typically remain hybridized to each other. Such stringent conditions are known to those skilled in the art and can be found in Current Protocols in Molecular Biology, John Wiley & Sons, N.Y. (1989), 6.3.1-6.3.6. A preferred, non-limiting example of stringent hybridization conditions are hybridization in 6× sodium chloride/sodium citrate (SSC) at about 45° C., followed by one or more washes in 0.2×SSC, 0.1% SDS at 50-65° C.

As used herein, the term “antibody” is intended to include immunoglobulin molecules and immunologically active portions of immunoglobulin molecules, i.e., molecules that contain an antigen binding site which specifically binds (immunoreacts with) an antigen, such as Fab and F(ab′)₂ fragments. The terms “monoclonal antibodies” and “monoclonal antibody composition”, as used herein, refer to a population of antibody molecules that contain only one species of an antigen binding site capable of immunoreacting with a particular epitope of an antigen, whereas the term “polyclonal antibodies” and “polyclonal antibody composition” refer to a population of antibody molecules that contain multiple species of antigen binding sites capable of interacting with a particular antigen. A monoclonal antibody compositions thus typically display a single binding affinity for a particular antigen with which it immunoreacts.

As used herein, the term “adjuvant” includes agents which potentiate the immune response to an antigen. Adjuvants can be administered in conjunction with the subject polypeptides to additionally augment the immune response.

As used herein, the term “enhancing an immune response” includes increasing T and/or B cell responses, i.e., cellular and/or humoral immune responses, by treatment of a subject using the claimed methods. In one embodiment, the claimed methods can be used to enhance T helper cell responses. In another embodiment, the claimed methods can be used to enhance cytotoxic T cell responses. The claimed methods can be used to enhance both primary and secondary immune responses. Preferably, the immune response is increased as compared to the response of immune cells to the antigen in the absence of treatment with the claimed methods. The immune response of a subject can be determined by, for example, assaying antibody production, immune cell proliferation, the release of cytokines, the expression of cell surface markers, cytotoxicity, enchanced ability to clear infection with HCV, etc.

As used herein, the term “particular clinical state” includes disorders or conditions associated with HCV infection, (e.g., cirrhosis, hepatocellular carcinoma) as well as clinical states associated with HCV infection, e.g., responsiveness or unresponsiveness to therapy for HCV.

II. Polypeptides Comprising an Amino Acid Sequence Encoded by an HCV Alternate Reading Frame

The polypeptides comprising an amino acid sequence encoded by an HCV alternate reading frame of the invention (e.g., naturally occurring polypeptides or consensus polypeptides) comprise an amino acid sequence which is not derived from an HCV polyprotein, i.e., the polypeptides of the present invention comprise an amino acid sequence encoded by an HCV alternate reading frame. An amino acid sequence encoded by an HCV alternate reading frame is not encoded by the standard HCV ORF; alternate reading frame polypeptides are translated from (or synthesized based on) a reading frame which is +1 or +2 to the standard HCV ORF. The position of the first nucleotide of an ORF in which these polypeptides are translated will vary slightly depending upon the isolate studied. For example, for the infectious clone (GenBank accession number AF011751) the first nucleotide of the ORF in which the polypeptides comprising an amino acid sequence encoded by an HCV alternate reading frame are translated is nucleotide 346, which is +5 relative to the standard HCV ORF. The first nucleotide of a codon of other, known or new isolates which results in a reading frame which corresponds to the reading frame of SEQ ID NO:1 can be determined, e.g., by performing a BLAST search using the nucleic acid sequence of SEQ ID NO:1 as the query sequence as described above.

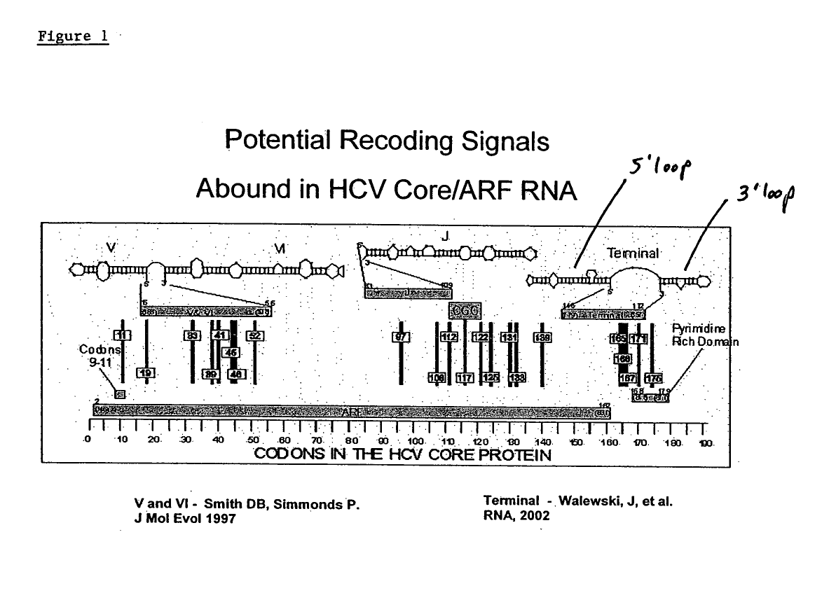

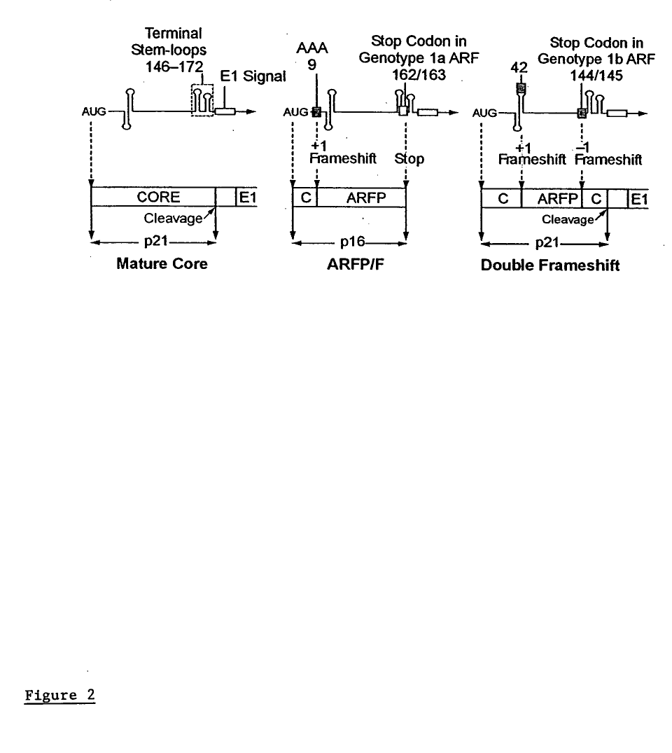

Translation of the polypeptides comprising an amino acid sequence encoded by an HCV alternate reading frame of the invention does not necessarily have to begin at a start AUG codon. For example, previous work has shown that the start AUG of HCV could be mutated to AUU or CUG with little effect on translation efficiency (Clarke, supra). Alternatively, RNA editing may be involved in generating an initiator codon. Translation of the polypeptides comprising an amino acid sequence encoded by an HCV alternate reading frame may also begin at the initiation site of the standard HCV ORF with a frame shift into a different reading frame. Finally, translation may be initiated 5′ of the AUG start codon of the standard ORF in any of the three reading frames, but shifted into the +1 reading frame (relative to the standard ORF) so as to yield production of peptides which are, in part, at least 60% identical to a portion of SEQ ID NO. 2. The internal ribosome entry site (IRES) is a complex RNA structural element that includes part of the 5′ untranslated region of HCV RNA and part of the adjacent coding region. It may induce frame shifting or translational by passing. Other potential recoding signals in HCV RNA have also been described (e.g., Smith and Simmonds 1997. J. Mol. Evol. 45:238; Walewski et al. 2002 RNA 8:557; FIG. 1). In addition, two potential recoding sites have been mapped (Layergne et al. 2002. 9^{th} International Meeting on HCV and Related Viruses. 9:42). The tip of Stem-loop VI may also be a frameshift signal. Exemplary schemes for production of alternate reading frame polypeptides and frameshift back into the standard ORF are shown in FIG. 2. Recoding signals and alternate reading frame polypeptides vary among different viral variants, subgenotypes, and genotypes.

In one embodiment, at least a portion of an alternate reading frame polypeptide is encoded by a reading frame corresponding to the reading frame of SEQ ID NO:1 in which the first nucleotide of SEQ ID NO:1 is the first nucleotide of a codon. This reading frame can encode a polypeptide of at least about 126 amino acids in length before a termination codon is reached. Table 1 presents an alignment of polypeptides comprising an amino acid sequence encoded by an HCV alternate reading frame which are encoded in this reading frame from various HCV isolates, along with a majority sequence derived using the Clustal method of sequence alignment. Stop codons appear in certain of the isolates after amino acid 126. However, translation may proceed beyond these stop codons. For example, in certain cases, these stop codons may be sequencing errors. Alternatively, readthrough can occur by mutation, altered transcription, RNA editing, frame shifting or ribosome slippage. Therefore, even in the polypeptides in which a stop codon appears, in certain embodiments of the invention, the HCV alternate reading frame polypeptide may be longer, i.e., translation may proceed past a termination codon. Therefore, in the case of, e.g., the infectious clone AF011751, translation of the polypeptide could terminate, for example, at position 163 or 186 of SEQ ID NO:2. When the polypeptides comprising an amino acid sequence encoded by an HCV alternate reading frame of the invention are synthesized, these stop codons may be ignored.

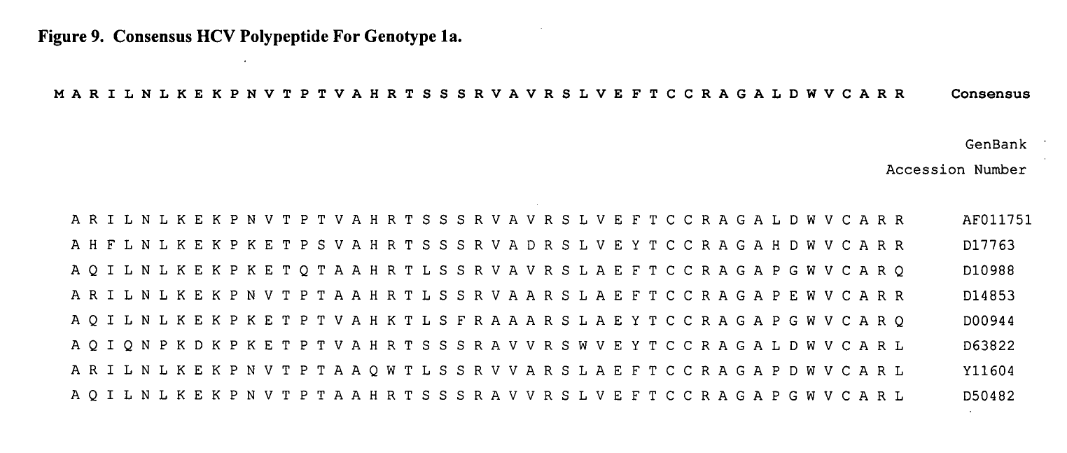

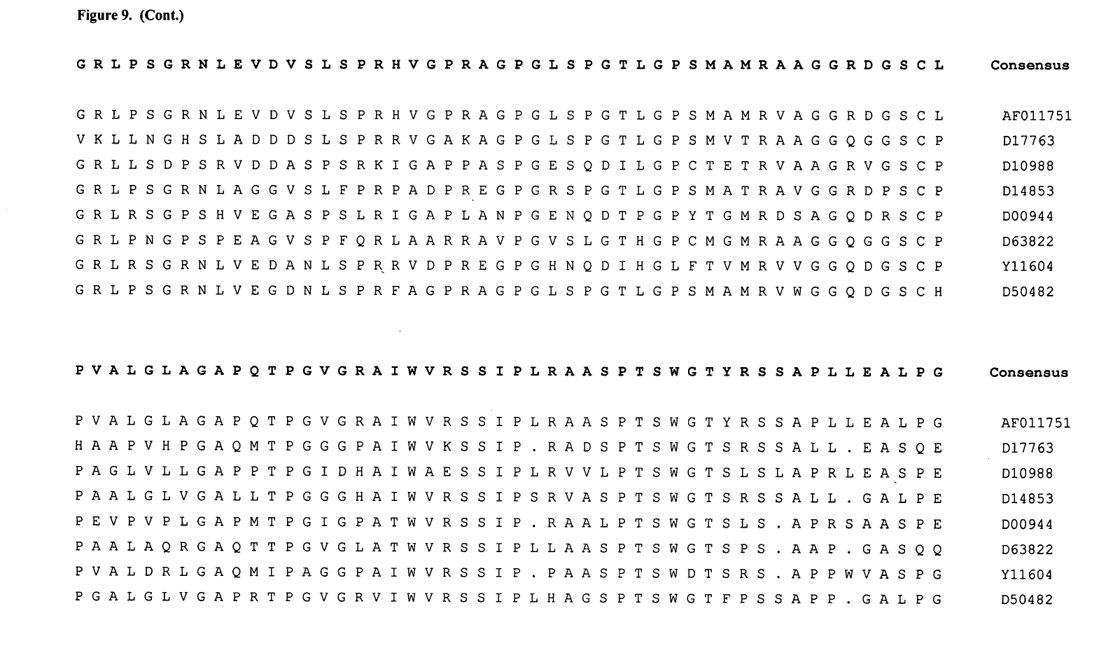

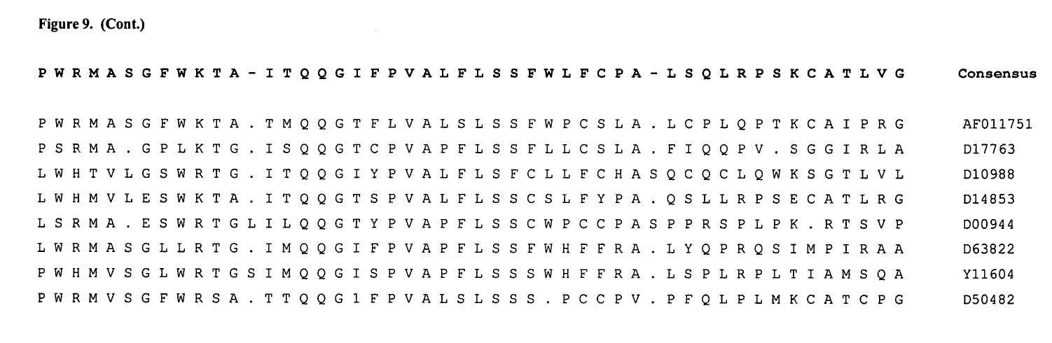

In other embodiments, a polypeptide of the invention comprises an amino acid sequence having some percentage identity to an alternate reading frame polypeptide amino acid sequence disclosed herein (e.g., as shown in SEQ ID NO:2, to an HCV alternate reading frame protein from another isolate, or to an alternate reading frame protein consensus sequence, e.g., as shown in FIG. 9). The percent identity between two nucleic acid or amino acid sequences can easily be calculated by dividing the number of identical bases or amino acids by the total number of bases or amino acids. Sequences are aligned to give the highest percent identity and yet provide an alignment which is biologically meaningful. Sequences can be aligned manually or, preferably, using an algorithm. For example, in the case of amino acid sequences, a FASTA search can be performed of the Swiss Protein database using the Biosum50.Cmp (scoring matrix). The gap creation penalty can be set, e.g., at 12 and the extension penalty can be set e.g., at 2. The joining threshold can be set, e.g., at 36; the optimization threshold can be set, e.g., at 24; and the optimization width can be set, e.g., at 16.

In certain embodiments, a HCV alternate reading frame polypeptide comprises an amino acid sequence at least about 40-50% identical to the amino acid sequence shown in the consensus sequence shown in FIG. 9. In preferred embodiments, the polypeptides comprising an amino acid sequence encoded by an HCV alternate reading frame comprise an amino acid sequence at least about 50-60% identical to the consensus sequence shown in FIG. 9. In other preferred embodiments, the polypeptides comprising an amino acid sequence encoded by an HCV alternate reading frame comprise an amino acid sequence at least about 60-70% identical to the consensus sequence shown in FIG. 9. In more preferred embodiments, the polypeptides comprising an amino acid sequence encoded by an HCV alternate reading frame comprise an amino acid sequence at least about 70-80% identical to the consensus sequence shown in FIG. 9. In still more preferred embodiments, the polypeptides comprising an amino acid sequence encoded by an HCV alternate reading frame comprise an amino acid sequence at least about 80-90% identical to the consensus sequence shown in FIG. 9. In other embodiments, a polypeptide of the invention comprises an amino acid sequence are identical to an alternate reading frame polypeptide amino acid sequence disclosed herein (e.g., as shown in SEQ ID NO:2, to an HCV alternate reading frame protein from another isolate, or to an alternate reading frame protein consensus sequence, e.g., as shown in FIG. 9).

In preferred embodiments, a polypeptide of the invention has the described percent identity over a length of at least about 10 amino acids. In more preferred embodiments, the percent identity of the polypeptides extends over a length of at least about 20-30 amino acids. In more preferred embodiment, the percent identity of the polypeptides extends over a length of at least about 30-40 amino acids. In a more preferred embodiment, the percent identity of the polypeptides extends over a length of at least about 40-50 amino acids. In another more preferred embodiment, the percent identity of the polypeptides extends over a length of more than 50 amino acids. In other preferred embodiments, the percent identity of the polypeptides extends over a length of more than 75 amino acids. In other preferred embodiments, the percent identity of the polypeptides extends over a length of more than 100 amino acids.

In other embodiments, a polypeptide of the invention comprises an amino acid sequence encoded by a nucleic acid molecule having some percentage identity to the nucleic acid molecule shown in SEQ ID NO:1, which is encoded in a reading frame +1 or +2 to the standard HCV reading frame. In certain embodiments, the polypeptides comprising an amino acid sequence encoded by an HCV alternate reading frame comprise an amino acid sequence encoded by a nucleic acid molecule at least 70% identical shown in SEQ ID NO:1 in which the polypeptide is encoded by the reading frame shown in SEQ ID NO:1. In preferred embodiments, the polypeptides comprising an amino acid sequence encoded by an HCV alternate reading frame comprises an amino acid sequence encoded by a nucleic acid molecule at least 80% identical shown in SEQ ID NO:1 in which the polypeptide is encoded by the reading frame shown in SEQ ID NO:1. In more preferred embodiments, the polypeptides comprising an amino acid sequence encoded by an HCV alternate reading frame comprise an amino acid sequence encoded by a nucleic acid molecule at least 90% identical shown in SEQ ID NO:1 in which the polypeptide is encoded by the reading frame shown in SEQ ID NO:1. In other embodiments, polypeptides comprising an amino acid sequence encoded by an HCV alternate reading frame comprise an amino acid sequence encoded by a nucleic acid molecule shown in SEQ ID NO:1 in which the polypeptide is encoded by the reading frame shown in SEQ ID NO:1.

In certain embodiments, a polypeptide of the invention is encoded by a nucleic acid molecule which hybridizes under stringent conditions to the nucleic acid sequence shown in SEQ ID NO:1 and in the reading frame shown in SEQ ID NO:1. Stringent hybridization conditions are known in the art. In preferred embodiments, such polypeptides are encoded by a nucleic acid molecule which hybridizes under stringent conditions to a nucleic acid molecule from any HCV isolate, but which are read in or synthesized as if read in the reading frame of SEQ ID NO: 1.

In one embodiment, a polypeptide of the invention is a chimeric polypeptide, comprising an amino acid sequence encoded by an HCV alternative reading frame and an amino acid sequence encoded by a standard HCV reading frame, e.g., core. For example, depending upon where the frameshift to the alternate reading frame occurs a naturally occurring alternate reading frame polypeptide may comprise amino acids derived from the standard HCV open reading frame.

In another embodiment, an alternate reading frame polypeptide of the invention is a chimeric polypeptide comprising an amino acid sequence encoded by an HCV alternate reading frame and a non-HCV amino acid sequence, e.g., a carrier protein sequence to enhance an immune response to the alternate reading frame amino acid sequence.

In one embodiment, a polypeptide of the invention is not a naturally occurring polypeptide, but rather is a consensus sequence derived from a comparison of naturally occurring alternate reading frame polypeptide sequences. To make a consensus polypeptide of the invention, a collection of alternate reading frame polypeptides from a number of genotypes can be aligned. The alternate reading frame proteins can be obtained, e.g., using the blast searching technique described above to identify sequences related to a query sequence, e.g., using SEQ ID NO:1. These sequences can be aligned and the ORF of other HCV isolates which corresponds to the standard HCV ORF of AF011751 (in which the initiation codon is at position 342, which is read as position 1 of the query sequence) can be read from the BLAST alignment. For example, the corresponding first nucleotide of a codon for GenBank accession no. HPCCGAA is 342. Another way to find the standard ORF would be to use a program, such as Edit Seq. (DNASTAR) which is designed to identify ORFS using the AUG aligned with position 342 of AF011751 as the start codon. Once the standard ORF is elucidated, the sequence encoding an alternate reading frame protein (e.g., +1 or +2 of the standard ORF) can be determined. The sequences can be readily translated into amino acid sequences to obtain the sequence of the alternate reading frame polypeptides.

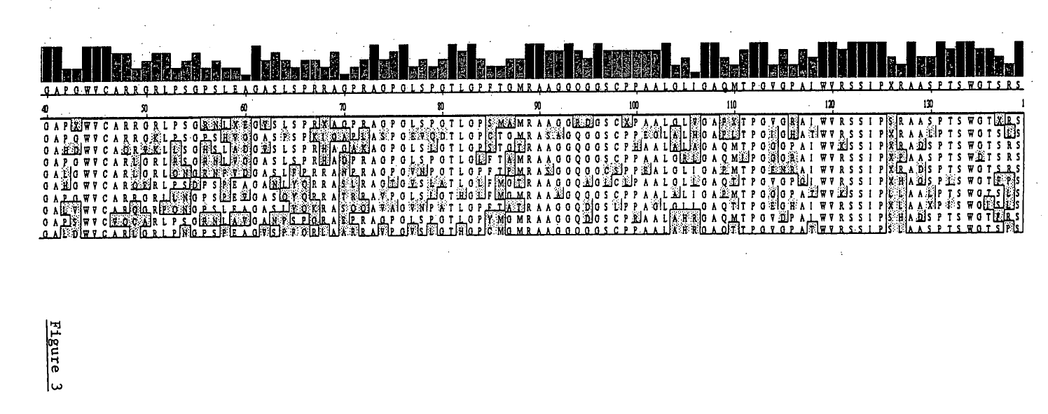

Once a collection of alternate reading frame polypeptides from each genotype is obtained, they can be aligned (e.g., using (MegAlign, DNAstar, Madison, Wis.) and used to generate a consensus sequence based on the simple majority of residues at each residue position. For example, the consensus alternate reading frame polypeptide sequences of genotypes 1-7 and 9-11 (genotype 8 sequences were excluded since there is no agreement on the position of the +1 stop codon) were aligned and displayed using the “alignment report” function of MegAlign (DNAstar). This alignment is shown in FIG. 3. Further refinements could come from generating consensus sequences based on the individual subtypes from each genotype (for example: 1a, 1b, 1c, 2a, 2b, 2c, 3a, 3b, 3c etc). At this level, there would be many more individual ARFP peptides. An example of a consensus sequence is shown in FIG. 9.

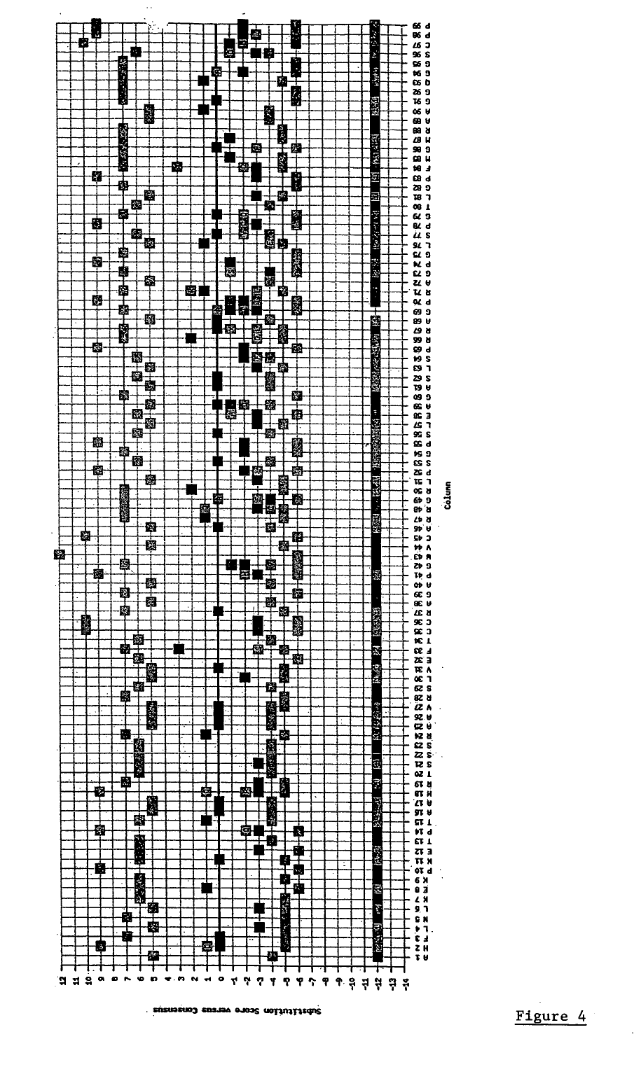

In another embodiment, the amino acid variation at each residue position in the collection of alternate reading frame polypeptide sequences could be analyzed by Mutation Master analysis (Walewski et al; 2002 RNA 8:557). This suite of programs allows extensive sequence alignments to compared to a “reference sequence”, and the incidence of all of the variant amino acids at each residue position can be tabulated, and scored for the degree of “conservation” using the BLOSUM tables for amino acids (Henikoff and Henifoff, 1992). An example of this is shown in FIG. 4. This can be used to indicate how representative a given reference sequence (e.g., a consensus sequence) is of a population of sequences and provides guidance as to where to vary the consensus.

In other embodiments, the polypeptides comprising an amino acid sequence encoded by an HCV alternate reading frame comprise at least a portion of an amino acid sequence selected from the group consisting of SEQ ID NO: 3, SEQ ID NO:4, SEQ ID NO:5, SEQ ID NO:6, SEQ ID NO:7, and SEQ ID NO:8 and cause an anti-HSV alternate reading frame immune response in a subject. Other polypeptides comprising an amino acid sequence encoded by an HCV alternate reading frame can be identified using an HCV nucleic acid sequence and determining the amino acids which are encoded in the +1 or +2 reading frame. Polypeptides comprising these sequences can be made and assayed for reactivity with antibodies from infected subjects. Those polypeptides which bind to antibodies, i.e., have elicited an immune response in infected subjects are made by the virus during the course of infection and represent preferred polypeptides comprising an amino acid sequence encoded by an HCV alternate reading frame.

In still other embodiments, the invention pertains to isolated or recombinant polypeptides comprising an amino acid sequence selected from the group consisting of: LNLKEKP(X1)(X2)TPT(X3) and AAHRT(X4)SSR(X5)(X6)VR, wherein X1 is N or K, X2 is V or E, X3 is A or V, X4 is L or S, X5 is A or V, and X6 is A or V. In yet other embodiments, the polypeptides comprising an amino acid sequence encoded by an HCV alternate reading frame consist of an amino acid sequence selected from the group consisting of LNLKEKPNVTPTA, AAHRTSSSRAVVR, and RAGAPGWVCARLGRLPSGR.

In another embodiment, a polypeptide of the invention comprises an amino acid sequence selected from the group consisting of:
GALDWVCARRGRLPSGRNLEVDVSLSPRHVGPRAGPGLSPGTLGPSMAMRAAG GRDGSCLPVALGLAGAPQTPGVGRAIWVRSSIPLRAASPTSWGTYRS;
GALDWVCARRGRLPSGRNLEVDVSLSPRHVGPRAGPGLSPGTLGPSMAMRAAG GRDGSCLPVALGLAGAPQTPGVGRAIWVRSSIPLRAASPTSWGTYRS; and
GALDWVCARRGRLPSGRNLEVDVSLSPRHVGPRAGPGLSPGTLGPSMAMRAAG GRDGSCLPVALGLAGAPQTPGVGRAIWVRSSIPLRAASPTSWGTYRS or immunogenic or immunoreactive portion thereof.

In another embodiment, a polypeptide of the invention comprises consensus amino acid sequence shown in FIG. 9, MARILNLKEKPNVTPTVAHRTSSSRVAVRSLVEFTCCRAGALDWVCARRGRLPS GRNLEVDVSLSPRHVGPRAGPGLSPGTLGPSMAMRAAGGRDGSCLPVALGLAG APQTPGVGRAIWVRSSIPLRAASPTSWGTYRSSAPLLEALPGPWRMASGFWKT, or immunogenic or immunoreactive portion thereof.

In another embodiment, a polypeptide of the invention comprises at least one amino acid sequence selected from the group consisting of:

| | |
|---|---|
| MARILNLKEKPNVTPTVA; | PNVTPTVAHRTSSSRVAV; |
| TSSSRVAVRSLVEFTCCR; | LVEFTCCRAGALDWVCAR; |
| ALDWVCARRGRLPSGRNL; | RLPSGRNLEVDVSLSPRH; |
| DVSLSPRHVGPRAGPGL; | GPRAGPGLSPGTLGPSMA; |
| GTLGPSMAMRAAGGRDGS; | AAGGRDGSCLPVALGLA; |
| LPVALGLAGAPQTPGVGR; | PQTPGVGRAIWVRSSIPL; |
| WVRSSIPLRAASPTSWGTY; | SPTSWGTYRSSAPLLEAL; |
| SAPLLEALPGPWRMASGF; | |
| and | |
| RMASGFPWRMASGFWKTA. | |

In another embodiment, a polypeptide of the invention comprises an amino acid sequence selected from the group consisting of:

MARILNLKEKPNVTPTVAHRTSSSRVAVRSLVEFTCCRAGALDWVCARRG
RLPSGRNLEVDVSLSPRHVG;
CARRGRLPSGRNLEVDVSLSRPHVGPRAGPGLSPGTLGPSMAMRAAGGRD
GSCLPVALGLAGAPQTPGVG;
and
GGRDGSCLPVALGLAGAPQTPGVGRAIWVRSSIPLRAASPTSWGTYRSSA
PLLEALPGPWRMASGFWKTA.

In one embodiment, a polypeptide of the invention is immobilized to a solid support.

Polypeptides can be modified using art recognized techniques to facilitate their attachment to a surface. In one embodiment, a carboxy terminal cysteine can be added to a polypeptide of the invention allow covalent linkage, e.g., to a BSA carrier or to a modified substrate (microbead or plastic plate).

In certain embodiments of the invention the polypeptides comprising an amino acid sequence encoded by an HCV alternate reading frame of the invention are at least about 10 amino acids to at least about 200 amino acids in length. In other embodiments, the polypeptides of the invention are at least about 20 amino acids to at least about 180 amino acids in length. In other embodiments, the polypeptides of the invention are at least about 30 amino acids to at least about 170 amino acids in length In other embodiments, the polypeptides of the invention are at least about 40 to at least about 160 amino acids in length. In other embodiments, the polypeptides of the invention are at least about 50 amino acids to at least about 150 amino acids in length. In other embodiments, the polypeptides of the invention are at least about 60 amino acids to at least about 140 amino acids in length. In other embodiments, the polypeptides of the invention are at least about 70 amino acids to at least about 130 amino acids in length. In other embodiments, the polypeptides of the invention are at least about 80 amino acids to at least about 120 amino acids in length. In other embodiments, the polypeptides of the invention are at least about 90 amino acids to at least about 110 amino acids in length. In another embodiment, the polypeptides of the invention are at least about 100 amino acids in length.

In preferred embodiments, the polypeptides comprising an amino acid sequence encoded by an HCV alternate reading frame are of a length sufficient to cause an anti-HCV alternate reading frame polypeptide immune response in a subject. Such an immune response can be measured using techniques which are known in the art. For example, the immune response elicited by the HCV polypeptides of the invention can be a T cell-mediated response which can be measured by, e.g., cytokine production and/or cellular proliferation or cellular cytotoxicity and/or a B cell mediated response which can be measured, e.g., by antibody production.

In another embodiment, an HCV alternate reading frame polypeptide is of a length sufficient to immunoreact with an antibody specific for an HCV alternate reading frame polypeptide as compared to an HCV standard reading frame polypeptide.

In certain embodiments the polypeptides comprising an amino acid sequence encoded by an HCV alternate reading frame of the invention are made as fusion proteins. In one embodiment, a fusion protein of the invention can range in length from about 50 to about 500 amino acids in length. In another embodiment, a fusion protein can range in length from about 100 to about 400 amino acids in length. In yet another embodiment, a fusion protein can range in length from about 200 to about 300 amino acids in length. In addition to utilizing fusion proteins to enhance immunogenicity, fusion proteins can also facilitate the expression of proteins, including the polypeptides comprising an amino acid sequence encoded by an HCV alternate reading frame of the present invention. For example, a the HCV alternate reading frame polypeptide can be fused to a non-HCV polypeptide, e.g., it can be generated as a glutathione-S-transferase (GST-fusion protein). Such GST-fusion proteins can enable easy purification of the HCV alternate reading frame polypeptide, as for example by the use of glutathione-derivatized matrices (see, for example, Current Protocols in Molecular Biology, eds. Ausubel et al. (N.Y.: John Wiley & Sons, 1991)). In another embodiment, a fusion gene coding for a purification leader sequence, such as a poly-(His)/enterokinase cleavage site sequence, can be can be fused to a the HCV alternate reading frame polypeptide, in order to permit purification of the poly(His)—the HCV alternate reading frame polypeptide by affinity chromatography using a Ni²⁺ metal resin. The purification leader sequence can then be subsequently removed by treatment with enterokinase (e.g., see Hochuli et al. (1987) J. Chromatography 411:177; and Janknecht et al. PNAS 88:8972).

Fusion proteins can also be used to enhance an immune response to an amino acid sequence encoded by an HCV alternate reading frame.

In one embodiment, a fusion protein of the invention comprises an alternate reading frame polypeptide fused to a polypeptide encoded by an HCV standard reading frame.

Techniques for making fusion genes are known to those skilled in the art. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed to generate a chimeric gene sequence (see, for example, Current Protocols in Molecular Biology, eds. Ausubel et al. John Wiley & Sons: 1992).

It will be understood that the preceding characteristics of HCV polypeptides are not mutually exclusive.

III. Production of Polypeptides Comprising an Amino Acid Sequence Encoded by an HCV Alternate Reading Frame

polypeptides comprising an amino acid sequence encoded by an HCV alternate reading frame can be produced by recombinant DNA techniques. For example, a nucleic acid molecule encoding such a polypeptide is cloned into an expression vector, the expression vector is introduced into a host cell and the HCV alternate reading frame polypeptide is expressed in the host cell. The HCV alternate reading frame polypeptide can then be isolated from the cells by an appropriate purification scheme using standard protein purification techniques. As an alternative to recombinant expression, a HCV alternate reading frame polypeptide can be synthesized chemically using standard peptide synthesis techniques or purchased commercially. Moreover, native polypeptides comprising an amino acid sequence encoded by an HCV alternate reading frame can be isolated from cells (e.g., cultured human cells infected with HCV), for example using an antibody.

A. Recombinant Production of HCV Alternate Reading Frame Polypeptides

In certain embodiments, the polypeptides comprising an amino acid sequence encoded by an HCV alternate reading frame are encoded by a naturally-occurring HCV nucleic acid molecule. As used herein, a “naturally-occurring” nucleic acid molecule refers to an RNA molecule (or a DNA molecule derived therefrom) having a nucleotide sequence that occurs in nature (e.g., encodes a protein produced by a naturally occurring HCV isolate).

In addition to naturally-occurring isolates of the polypeptides comprising an amino acid sequence encoded by an HCV alternate reading frame, the skilled artisan will further appreciate that changes may be introduced by mutation, e.g., into an HCV nucleotide sequence thereby leading to changes in the amino acid sequence of the encoded HCV polypeptides.

For example, an isolated nucleic acid molecule encoding a polypeptide comprising an amino acid sequence homologous to a consensus polypeptide or to the polypeptide of SEQ ID NO: 2, i.e., having a certain percentage identity to the consensus polypeptide or the polypeptide of SEQ ID NO:2 can be created by introducing one or more nucleotide substitutions, additions or deletions into the nucleotide sequence of SEQ ID NO: 1 such that one or more amino acid substitutions, additions or deletions are introduced into the encoded polypeptide. Mutations can be introduced into SEQ ID NO: 1 by standard techniques, such as site-directed mutagenesis and PCR-mediated mutagenesis. Alternatively, such a polypeptide can be chemically synthesized to yield a polypeptide with a change in amino acid sequence from that in the naturally occurring polypeptide.

Preferably, no substitutions or conservative amino acid substitutions are made where the is high homology or identity in amino acid residues among the various isolates as can be seen in a sequence alignment as shown in FIG. 9. A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

Alternatively, in another embodiment, mutations can be introduced randomly along all or part of a HCV alternate reading frame polypeptide coding sequence, such as by saturation mutagenesis, and the resultant mutants can be screened, e.g., by testing for reactivity with antibodies from an individual with a past or present HCV infection.

B. Expression Vectors and Host Cells

The nucleic acid molecules described herein can be expressed in an expression vector to produce a HCV alternate reading frame polypeptide. As used herein, the term “vector” refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid molecule as described herein in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to includes promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector may depend on such factors as the choice of the host cell to be transformed, the level of expression of protein desired, etc. The expression vectors of the invention can be introduced into host cells to thereby produce polypeptides comprising an amino acid sequence encoded by an HCV alternate reading frame, including fusion proteins comprising such polypeptides.

The recombinant expression vectors of the invention can be designed for expression of polypeptides comprising an amino acid sequence encoded by an HCV alternate reading frame in prokaryotic or eukaryotic cells. For example, polypeptides comprising an amino acid sequence encoded by an HCV alternate reading frame can be expressed in bacterial cells such as E. coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector may be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Examples of suitable inducible non-fusion E. coli expression vectors include pTrc (Amann et et al., (1988) Gene 69:301-315) and pET 11d (Studier et al, Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HMS174(DE3) from a resident λ prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, S., Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al., (1992) Nuc. Acids Res. 20:2111-2118). Such alteration of nucleic acid sequences of the invention can be carried out by standard DNA synthesis techniques.

In another embodiment, the polypeptides comprising an amino acid sequence encoded by an HCV alternate reading frame expression vector is a yeast expression vector. Examples of vectors for expression in yeast S. cerivisae include pYepSec1 (Baldari. et al., (1987) Embo J 6:229-234), pMFa (Kurjan and Herskowitz, (1982) Cell 30:933-943), pJRY88 (Schultz et al., (1987) Gene 54:113-123), and pYES2 (Invitrogen Corporation, San Diego, Calif.).

Alternatively, polypeptides comprising an amino acid sequence encoded by an HCV alternate reading frame can be expressed in insect cells using baculovirus expression vectors. Baculovirus vectors available for expression of proteins in cultured insect cells (e.g., Sf 9 cells) include the pAc series (Smith et al., (1983) Mol. Cell Biol. 3:2156-2165) and the pVL series (Lucklow, V. A., and Summers, M. D., (1989) Virology 170:31-39).

In yet another embodiment, a nucleic acid molecule encoding polypeptides comprising an amino acid sequence encoded by an HCV alternate reading frame of the invention is expressed in mammalian cells using a mammalian expression vector. Examples of mammalian expression vectors include pCDM8 (Seed, B., (1987) Nature 329:840) and pMT2PC (Kaufman et al. (1987), EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the α-fetoprotein promoter (Campes and Tilghman (1989) Genes Dev. 3:537-546).

A recombinant expression vector is introduced into a suitable host cell. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell may be any prokaryotic or eukaryotic cell. For example, polypeptides comprising an amino acid sequence encoded by an HCV alternate reading frame may be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook et al. (Molecular Cloning: A Laboratory Manual, 2nd Edition, Cold Spring Harbor Laboratory press (1989)), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker may be introduced into a host cell on the same vector as that encoding the polypeptide or may be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) polypeptides comprising an amino acid sequence encoded by an HCV alternate reading frame. Accordingly, the invention further provides methods for producing polypeptides comprising an amino acid sequence encoded by an HCV alternate reading frame using these host cells. In one embodiment, the method comprises culturing the host cell of invention (into which a recombinant expression vector encoding polypeptides comprising an amino acid sequence encoded by an HCV alternate reading frame has been introduced) in a suitable medium until a HCV alternate reading frame polypeptide is produced. In another embodiment, the method further comprises isolating polypeptides comprising an amino acid sequence encoded by an HCV alternate reading frame from the medium or the host cell.

C. Chemical Synthesis of Polypeptides Comprising an Amino Acid Sequence Encoded by an HCV Alternate Reading Frame

The polypeptides comprising an amino acid sequence encoded by an HCV alternate reading frame can be chemically synthesized as is well known in the art.

D. Modified Forms of Polypeptides

In one embodiment, the peptide can be substituted and/or derivatized to optimize stability or attachment to surfaces or to additional polypeptides using techniques well known in the art. The subject polypeptides can also be synthesized as branched polypeptides, particularly for vaccine applications as is known in the art (see, e.g., Peptides. Edited by Bernd Gutte Academic Press 1995. pp. 456-493).

IV. Antibodies which React with Polypeptides Comprising an Amino Acid Sequence Encoded by an HCV Alternate Reading Frame

In yet another aspect, the invention pertains to an antibody which binds to a HCV alternate reading frame polypeptide. A HCV alternate reading frame polypeptide, or fragment thereof, can be used as an immunogen to generate antibodies that bind such a polypeptide using standard techniques for polyclonal and monoclonal antibody preparation. The invention provides numerous antigenic peptide fragments of polypeptides comprising an amino acid sequence encoded by an HCV alternate reading frame for use as immunogens. Preferably, an antigenic peptide of such a polypeptide comprises at least 8 amino acid residues of an amino acid sequence encoded by an HCV alternate reading frame as described herein, e.g., of the amino acid sequence shown in SEQ ID NO: 2 or the consensus sequence shown in FIG. 9. Preferably, the antigenic peptide comprises at least 10 amino acid residues, more preferably at least 14 amino acid residues, even more preferably at least 18 amino acid residues. Any of the polypeptides described herein can be used. Exemplary polypeptides are described herein and comprise, e.g., the ARF #1 consensus sequence :LNLKEKP(X1)(X2)TPT(X3) or the ARF#2 consensus sequence AAHRT(X4)SSR(X5)(X6)VR, wherein X1 is N or K, X2 is V or E, X3 is A or V, X4 is L or S, X5 is A or V, and X6 is A or V polypeptide sequences. Other preferred HCV polypeptides comprise or consist of the sequence LNLKEKPNVTPTA or AAHRTSSSRAVVR.

The subject HCV polypeptides can be used to prepare antibodies by immunizing a suitable subject, (e.g., rabbit, goat, mouse or other mammal) with the immunogen using art-recognized techniques. An appropriate immunogenic preparation can contain, for example, recombinantly expressed polypeptides comprising an amino acid sequence encoded by an HCV alternate reading frame or a chemically synthesized HCV alternate reading frame polypeptide can be used. The preparation can further include an adjuvant, such as Freund's complete or incomplete adjuvant, or similar immunostimulatory agent. Immunization of a suitable subject with an immunogenic HCV polypeptides preparation induces a polyclonal HCV polypeptides antibody response.

Accordingly, another aspect of the invention pertains to antibodies which react with the polypeptides comprising an amino acid sequence encoded by an HCV alternate reading frame. The term “antibody” as used herein refers to immunoglobulin molecules and immunologically active portions of immunoglobulin molecules, i.e., molecules that contain an antigen binding site which specifically binds (immunoreacts with) an HCV polypeptides. The invention provides polyclonal and monoclonal antibodies that bind HCV polypeptides. The term “monoclonal antibody” or “monoclonal antibody composition”, as used herein, refers to a population of antibody molecules that contain only one species of an antigen binding site capable of immunoreacting with a particular epitope of a HCV alternate reading frame polypeptide. A monoclonal antibody composition thus typically displays a single binding affinity for a particular HCV polypeptide with which it reacts.

Polyclonal or monoclonal anti-HCV polypeptide antibodies can be prepared as described above by immunizing a suitable subject with a HCV polypeptide immunogen or attenuated HCV virus, or can be obtained from an infected individual. The anti-HCV polypeptide antibody titer in the immunized subject can be monitored over time by standard techniques, such as with an enzyme linked immunosorbent assay (ELISA) using immobilized HCV polypeptide. If desired, the antibody molecules directed against HCV polypeptide can be isolated from the animal (e.g., from the blood) and further purified by well known techniques, such as protein A chromatography to obtain the IgG fraction. At an appropriate time after immunization, e.g., when the antibody titers are highest, antibody-producing cells can be obtained from the subject and used to prepare monoclonal antibodies by standard techniques, such as the hybridoma technique originally described by Kohler and Milstein (1975, Nature 256:495-497) (see also, Brown et al. (1981) J. Immunol 127:539-46; Brown et al. (1980) J Biol. Chem 255:4980-83; Yeh et al. (1976) PNAS 76:2927-31; and Yeh et al. (1982) Int. J. Cancer 29:269-75), the more recent human B cell hybridoma technique (Kozbor et al. (1983) Immunol Today 4:72), the EBV-hybridoma technique (Cole et al. (1985), Monoclonal Antibodies and Cancer Therapy, Alan R. Liss, Inc., pp. 77-96) or trioma techniques. The technology for producing monoclonal antibody hybridomas is well known (see generally R. H. Kenneth, in Monoclonal Antibodies: A New Dimension In Biological Analyses, Plenum Publishing Corp., New York, N.Y. (1980); E. A. Lerner (1981) Yale J. Biol. Med, 54:387-402; M. L. Gefter et al. (1977) Somatic Cell Genet., 3:231-36). Briefly, an immortal cell line (typically a myeloma) is fused to lymphocytes (typically splenocytes) from a mammal immunized with a HCV polypeptide immunogen as described above, and the culture supernatants of the resulting hybridoma cells are screened to identify a hybridoma producing a monoclonal antibody that binds the HCV polypeptide.

Any of the many well known protocols used for fusing lymphocytes and immortalized cell lines can be applied for the purpose of generating an anti-HCV polypeptide monoclonal antibody (see, e.g., G. Galfre et al. (1977) Nature 266:55052; Gefter et al. Somatic Cell Genet., cited supra; Lerner, Yale J. Biol. Med., cited supra; Kenneth, Monoclonal Antibodies, cited supra). Moreover, the ordinary skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines may be used as a fusion partner according to standard techniques, e.g., the P3-NS1/1-Ag4-1, P3-x63-Ag8.653 or Sp2/O-Ag14 myeloma lines. These myeloma lines are available from the American Type Culture Collection (ATCC), Rockville, Md. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed).

Hybridoma cells producing a monoclonal antibody of the invention are detected by screening the hybridoma culture supernatants for antibodies that bind one or more amino acid sequences encoded by an HCV alternate reading frame, e.g., using a standard ELISA assay.

Alternative to preparing monoclonal antibody-secreting hybridomas, a monoclonal anti-HCV polypeptide antibody can be identified and isolated by screening a recombinant combinatorial immunoglobulin library (e.g., an antibody phage display library) with HCV polypeptides to thereby isolate immunoglobulin library members that bind HCV polypeptides. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Bio/Technology 9:1370-1372; Hay et al. (1992) Hum Antibod Hybridomas 3:81-85; Huse et al. (1989) Science 246:1275-1281; Griffiths et al. (1993) EMBO J 12:725-734; Hawkins et al. (1992) J Mol Biol 226:889-896; Clarkson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; Barbas et al. (1991) PNAS 88:7978-7982; and McCafferty et al. Nature (1990) 348:552-554.

Several publications (e.g., Marks et al. Bio/Technology 10:779-783 (1992)) have described the production of high affinity human antibodies by chain shuffling, as well as combinatorial infection and in vivo recombination as a strategy for constructing large phage libraries. In another embodiment, Ribosomal display can be used to replace bacteriophage as the display platform (see, e.g., Hanes et al. 2000. Nat. Biotechnol. 18:1287; Wilson et al. 2001. Proc. Natl. Acad. Sci. USA 98:3750; or Irving et al. 2001 J. Immunol. Methods 248:31. In yet another embodiment, cell surface libraries can be screened for antibodies (Boder et al. 2000. Proc. Natl. Acad. Sci. USA 97:10701; Daugherty et al. 2000 J. Immunol. Methods 243:211. Such procedures provide alternatives to traditional hybridoma techniques for the isolation and subsequent cloning of monoclonal antibodies.

Yet other embodiments of the present invention comprise the generation of human or substantially human antibodies in transgenic animals (e.g., mice) that are incapable of endogenous immunoglobulin production (see e.g., U.S. Pat. Nos. 6,075,181, 5,939,598, 5,591,669 and 5,589,369 each of which is incorporated herein by reference). For example, it has been described that the homozygous deletion of the antibody heavy-chain joining region in chimeric and germ-line mutant mice results in complete inhibition of endogenous antibody production. Transfer of a human immunoglobulin gene array to such germ line mutant mice will result in the production of human antibodies upon antigen challenge. Another preferred means of generating human antibodies using SCID mice is disclosed in U.S. Pat. No. 5,811,524 which is incorporated herein by reference. It will be appreciated that the genetic material associated with these human antibodies may also be isolated and manipulated as described herein. Such human antibodies are particularly suited for passive immunotherapy.

Yet another highly efficient means for generating recombinant antibodies is disclosed by Newman, Biotechnology, 10: 1455-1460 (1992). Specifically, this technique results in the generation of primatized antibodies that contain monkey variable domains and human constant sequences. This reference is incorporated by reference in its entirety herein. Moreover, this technique is also described in commonly assigned U.S. Pat. Nos. 5,658,570, 5,693,780 and 5,756,096 each of which is incorporated herein by reference.

In another embodiment, lymphocytes can be selected by micromanipulation and the variable genes isolated. For example, peripheral blood mononuclear cells can be isolated from an immunized mammal and cultured for about 7 days in vitro. The cultures can be screened for specific IgGs that meet the screening criteria. Cells from positive wells can be isolated. Individual Ig-producing B cells can be isolated by FACS or by identifying them in a complement-mediated hemolytic plaque assay. Ig-producing B cells can be micromanipulated into a tube and the VH and VL genes can be amplified using, e.g., RT-PCR. The VH and VL genes can be cloned into an antibody expression vector and transfected into cells (e.g., eukaryotic or prokaryotic cells) for expression. Additionally, recombinant anti-HCV polypeptide antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in Robinson et al. International Patent Publication PCT/US86/02269; Akira, et al. European Patent Application 184,187; Taniguchi, M., European Patent Application 171,496; Morrison et al. European Patent Application 173,494; Neuberger et al. PCT Application WO 86/01533; Cabilly et al. U.S. Pat. No. 4,816,567; Cabilly et al. European Patent Application 125,023; Better et al. (1988) Science 240:1041-1043; Liu et al. (1987) PNAS 84:3439-3443; Liu et al. (1987) J. Immunol. 139:3521-3526; Sun et al. (1987) PNAS 84:214-218; Nishimura et al. (1987) Canc. Res. 47:999-1005; Wood et al. (1985) Nature 314:446-449; and Shaw et al. (1988) J. Natl Cancer Inst. 80:1553-1559); Morrison, S. L. (1985) Science 229:1202-1207; Oi et al. (1986) BioTechniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:4053-4060.

An anti-HCV polypeptide antibody (e.g., monoclonal antibody) can be used to isolate or detect HCV polypeptides by standard techniques, such as affinity chromatography, immunoprecipitation, ELISA, in situ hybridization, or RIA as is well known in the art. An anti-HCV polypeptide antibody can facilitate the purification of natural HCV polypeptide from cells and of recombinantly produced HCV polypeptides expressed in host cells. Moreover, an anti-HCV polypeptide antibody can be used to detect HCV polypeptides from cells or body fluid of a subject which is suspected to have an HCV infection. Detection may be facilitated by coupling (i.e., physically linking) the antibody to a detectable substance. Examples of detectable substances include various enzymes, prosthetic groups, fluorescent materials, luminescent materials and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; and examples of suitable radioactive material include ¹²⁵I, ¹³¹I, ³⁵S or ³H.

Such antibodies can be incorporated in diagnostic kits and are also useful in passive immunization against HCV in patients which have an active HCV infection or are likely to be exposed to HCV.

V. Uses of Polypeptides Comprising an Amino Acid Sequence Encoded by an HCV Alternate Reading Frame

In another aspect, the invention pertains to a vaccine composition which is administered to a subject prior to exposure to HCV to preventing hepatitis C infection in the subject or during infection with HCV to facilitate viral clearance or to reduce the likelihood of progressing to chronic infection or liver cancer. In one embodiment, the vaccine comprises a polypeptide comprising an amino acid sequence encoded by an HCV alternate reading frame. In another embodiment, the vaccine causes an HCV alternate reading frame polypeptide of the invention to be synthesized in a subject.

A. Vaccines

HCV alternate reading frame polypeptide sequences appropriate for use in vaccine compositions for the prevention of HCV in a subject can easily be determined. For example, epitopes which elicit an immune response can be identified by screening in an immunoassay against sera from patients with a past or ongoing HCV infection. Alternatively, immunogenic polypeptides can be identified by computer analysis to identify immunogenic epitopes. Finally, the full-length novel polypeptide could be used in a vaccine.

In another embodiment, agents which are known adjuvants can be administered with the subject polypeptides. At this time, the only adjuvant widely used in humans has been alum (aluminum phosphate or aluminum hydroxide). Saponin and its purified component Quil A, Freund's complete adjuvant and other adjuvants used in research and veterinary applications have potential use in human vaccines. However, new chemically defined preparations such as muramyl dipeptide, monophosphoryl lipid A, phospholipid conjugates such as those described by Goodman-Snitkoff et al. J. Immunol. 147:410-415 (1991) resorcinols, non-ionic surfactants such as polyoxyethylene oleyl ether and n-hexadecyl polyethylene ether, enzyme inhibitors include pancreatic trypsin inhibitor, diisopropylfluorophosphate (DEP) and trasylol can also be used. In embodiments in which antigen is administered, the antigen can e.g., be encapsulated within a proteoliposome as described by Miller et al., J. Exp. Med. 176:1739-1744 (1992) and incorporated by reference herein, or in lipid vesicles, such as Novasome TM lipid vesicles (Micro Vescular Systems, Inc., Nashua, N.H.), to further enhance immune responses.

In yet other embodiments, as an alternative to administering the HCV alternate reading frame polypeptide, the polypeptide can be synthesized by the subject. This can be done using a plasmid DNA construct which is similar to those used for delivery of reporter or therapeutic genes. Such a construct preferably comprises a bacterial origin of replication that allows amplification of large quantities of the plasmid DNA; a prokaryotic selectable marker gene; a nucleic acid sequence encoding a HCV alternate reading frame polypeptide or portion thereof; eukaryotic transcription regulatory elements to direct gene expression in the host cell; and a polyadenylation sequence to ensure appropriate termination of the expressed mRNA (Davis. 1997. Curr. Opin. Biotechnol. 8:635). Vectors used for DNA immunization may optionally comprise a signal sequence (Michel et al. 1995. Proc. Natl. Acad Sci USA. 92:5307; Donnelly et al. 1996. J. Infect Dis. 173:314). DNA vaccines can be administered by a variety of means, for example, by injection (e.g., intramuscular, intradermal, or the biolistic injection of DNA-coated gold particles into the epidermis with a gene gun that uses a particle accelerator or a compressed gas to inject the particles into the skin (Haynes et al. 1996. J. Biotechnol. 44:37)). Alternatively, DNA vaccines can be administered by non-invasive means. For example, pure or lipid-formulated DNA can be delivered to the respiratory system or targeted elsewhere, e.g., Peyers patches by oral delivery of DNA (Schubbert. 1997. Proc. Natl. Acad. Sci. USA 94:961). Attenuated microorganisms can be used for delivery to mucosal surfaces. (Sizemore et al. 1995. Science. 270:29).

Any of the instant vaccine compositions can comprise (or encode) one or more epitopes (either contiguous or non contiguous) of a HCV alternate reading frame polypeptide of the invention. Such preparations can further comprise polypeptide sequences derived from an HCV polyprotein sequence, e.g., from core.

In another embodiment, a vaccine composition can comprise more than one polypeptide. For example, two alternate reading frame polypeptides, two consensus polypeptides, an alternate reading frame polypeptide and a consensus polypeptide, or a consensus polypeptide and a standard reading frame polypeptide, etc. can be included.

In other embodiments, such a vaccine composition can further comprise a compound which will enhance the immunologocial reactivity of the HCV alternate reading frame polypeptide epitope. For example, the immunogenicicty of the polypeptides comprising an amino acid sequence encoded by an HCV alternate reading frame may be enhanced by making a fusion proteins comprising a HCV alternate reading frame polypeptide fused to a different polypeptide, i.e., not a HCV alternate reading frame polypeptide. Techniques for making such fusion proteins are known in the art. Alternatively, a vaccine can comprise an immunoregulatory molecule, such as a cytokine. For example, in one embodiment, plasmids for DNA vaccination can express a single immunogen, or two sequences can be coexpressed. In one embodiment, the additional sequences can be additional immunogens (polypeptides comprising an amino acid sequence encoded by an HCV alternate reading frame or HCV polyprotein polypeptides or other polypeptides) or can encode modulators of immune responses such as lymphokine genes or costimulatory molecules (Iwasaki et al. 1997. J. Immunol. 158:4591).

Typically, vaccine compositions of the present invention are prepared as injectables, either as liquid solutions or suspensions. Solid forms suitable for solution in, or suspension in, liquid prior to injection may also be prepared. The composition may also be emulsified, or the polypeptide encapsulated into liposomes. The polypeptide may be mixed with pharmaceutically acceptable excipients, for example, water, saline, dextrose, glycerol, ethanol, or the like. The composition may also comprise minor amounts of, for example, wetting agents, pH buffering agents and/or adjuvants, such as aluminum hydroxide, N-acetyl-muramyl-L-threonyl-D-isoglutamine (thr-MDP), N-acetyl-nor-muramyl-L-alanyl-D-isoglutamine (CGP 11637 or nor-MDP), N-acetylmuramlyl-L-alanyl-D-isoglutaminyl-L-alanine-2-(1′-2′-dipalmitoyl-sn-glycerol-3-hydroxyphosphoryloxy)ethylamine (CGP 19835A, or MTP-PE), or bacterial components.

Such vaccine compositions are generally administered parenterally, by injection, usually wither subcutaneously or intramuscularly. Other formulations may be administered orally, by inhalation or as suppositories.

The polypeptides may be incorporated into the vaccine in a neutral or salt form. Pharmaceutically acceptable salts include the acid addition salts (formed with free amino groups of the polypeptide) and which are formed with inorganic acids, such as, for example, hydrochloric or phosphoric acids, or such organic acids such as acetic, oxalic, tartaric, maleic, or the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, or the like.

The vaccines are administered so as to be compatible with the dosage formulation, and in such an amount as will be prophylactically and/or therapeutically effective. The quantity to be administered depends on the subject to be treated, the capacity of the subject's immune system to mount an immune response to the vaccine, and the degree of protection desired. The range of 5 μg to 250 μg of antigen per dose, however, is often appropriate. The vaccine compositions may be given in a single dose or in multiple doses. Appropriate doses are well within the skill of the art to determine, and do not constitute undue experimentation.

In still another embodiment, the invention pertains to a method of preventing HCV in a subject by administering a HCV alternate reading frame polypeptide to a subject or by causing a HCV alternate reading frame polypeptide to be expressed in a subject, e.g., by administration of a DNA vaccine.

In one embodiment, a vaccine of the invention may be administered in conjunction with traditional anti-viral therapy, e.g., IFN alpha (e.g., pegylated or non-pegylated) or IFN alpha and ribavirin.

B. Use in Diagnostic or Prognostic Assays and Kits

In another aspect of the invention, methods for diagnosing HCV infection e.g., either a past or present infection, and/or a disease state caused by HCV and diagnostic kits are provided for detecting an infection with HCV and/or a disease state caused by HCV.

In one embodiment, the invention provides a method of diagnosing HCV infection (or a particular clinical state caused by HCV) by detecting an immune response to a polypeptide comprising an amino acid sequence encoded by an HCV alternate reading frame in a subject, e.g., a B cell response (as detected by antibodies) or a T cell response (e.g., a CTL or helper T cell response as detected using in vitro assays such as proliferation).

In one embodiment, the invention provides a method of diagnosing HCV infection by detecting the presence or absence of antibodies in the body fluid of a subject which bind to an HCV alternate reading frame amino acid sequence. In one embodiment the method comprises incubating a test sample under conditions which allow the binding of a HCV alternate reading frame polypeptide and an antibody in the test sample of body fluid and detecting the binding of polypeptide and antibody.

Test samples can be derived from any appropriate body fluid or tissue preparation, for example, whole blood, plasma, serum, spinal fluid, lymph fluid, tears, saliva, milk, or liver tissue preparations.

Detection of the binding between a HCV alternate reading frame polypeptide of the invention and an antibody can be accomplished using any technique which is known in the art and can be facilitated using antibodies labelled as described above.

Antibodies which bind to HCV alternate reading frame polypeptide can be detected using a number of different screening assays known in the art, such as an enzyme-linked immunosorbent assay (ELISA), a radioimmunoassay (RIA), or a Western Blot Assay. Each assay generally detects the presence of protein-antibody complexes of particular interest by employing a labeled reagent (e.g., an antibody) specific for the complex of interest. Accordingly, in the present invention, these assays are used to detect HCV alternate reading frame polypeptide-antibody complexes formed between immunoglobulins (e.g., human IgG, IgM and IgA) contained in a biological sample and a HCV alternate reading frame polypeptide.

In one embodiment one or more of the polypeptides comprising an amino acid sequence encoded by an HCV alternate reading frame of the invention can be used to detect antibodies in a test sample. For example, two alternate reading frame polypeptides, two consensus polypeptides, an alternate reading frame polypeptide and a consensus polypeptide, or a consensus polypeptide and a standard reading frame polypeptide, etc. can be used to increase the likelihood of detecting antibody which recognizes one of the polypeptides.

In another embodiment, different forms of alternate reading frame polypeptides can be used depending upon the disorder to be detected. For instance, in one embodiment an alternate reading frame polypeptide can be derived from the amino terminal region, from the central domain, or from the carboxyterminal region of a polypeptide corresponding to the consensus sequence of FIG. 9. For example, in one embodiment, an alternate reading frame polypeptide derived from the amino terminal region of an alternate reading frame polypeptide (e.g., as shown in the consensus sequence in FIG. 9) is used to detect acute infection or to examine reactivity of a subject being treated with an anti-HSV therapy (e.g., with interferon (IFN) or interferon plus ribavirin). In another embodiment, an alternate reading frame polypeptide derived from the middle region of an alternate reading frame polypeptide (e.g., as shown in the consensus sequence in FIG. 9) is used to detect a patient which has, or is at risk for developing, hepatocellular carcinoma.

As will be described below, protein-antibody complexes formed during the detection process are preferably detected using an enzyme-linked antibody or antibody fragment (e.g., a monoclonal antibody or fragment thereof) which recognizes and specifically binds to the polypeptide-antibody complexes.

In one embodiment of the method, a sandwhich ELISA assay is used. For example, a HCV alternate reading frame polypeptide with or without conjugation to a carrier, such as activated BSA is immobilized on a plate. A body fluid sample from an individual is contacted with a HCV alternate reading frame polypeptide under conditions which allow binding of the antibodies in the sample to the polypeptides. The sample is then removed, and any antibody which has bound to the HCV polypeptide is detected by contacting the sample with a labeled secondary antibody or antibody fragment which binds to an antibody which might be present in the subjects sample, e.g., an anti-human antibody. The unbound secondary antibody is removed and the presence of secondary antibody which remains bound is detected, e.g., using a label as described above. Possible controls for use in the method include body fluids from uninfected subjects and polypeptides which are not polypeptides comprising an amino acid sequence encoded by an HCV alternate reading frame. In accordance with the present invention, the presence of such an antibody is indicative of an infection with HCV.

In another embodiment of the assay the test sample can be tested for the presence of polypeptides comprising an amino acid sequence encoded by an HCV alternate reading frame using known antibodies. In one embodiment, such a polypeptide can be detected by electron microscopy using techniques known in the art. In another embodiment, antibodies that bind to polypeptides comprising an amino acid sequence encoded by an HCV alternate reading frame are used to detect the presence of polypeptides comprising an amino acid sequence encoded by an HCV alternate reading frame in the body fluid of a subject or in a cell of a subject. In performing such an assay, the antibodies which bind to a HCV alternate reading frame polypeptide are contacted with a cell or body fluid of a subject under conditions where a HCV alternate reading frame polypeptide in the subject's sample can bind to the antibody. Unbound antibody is removed and bound antibody is detected. Any of the antibodies described above can be used in practicing this method. Preferred antibodies for use in the methods of this embodiment are highly specific for alternate reading frame amino acid sequences as compared to standard reading frame amino acid sequences, including monospecific and, more preferably, monoclonal antibodies or fragments thereof. In one embodiment, such antibodies are labelled. In other embodiments, such antibodies are detected by employing a secondary antibody which binds to them and not to the test sample from a subject. The presence of a HCV alternate reading frame polypeptide in the subject's sample is indicative of an infection with HCV. Viral polypeptides can be detected using methods known in the art.

In another embodiment, the invention pertains to a method of predicting the propensity of a patient infected with an HCV virus to clear HCV spontaneously, to clear HCV in response to IFN, to progress to chronic infection, to fail IFN treatment, to develop chronic HCV disease, cirrhosis, or liver cancer comprising detecting sequence changes in alternate reading frame polypeptides made by the virus. For example, in one embodiment, the presence or absence of antibodies directed against specific alternate reading frame protein regions (epitopes) in a subject can be detected to determine the prognosis of the patient or the best course for continued treatment. In one embodiment, patients with advanced cirrhosis and hepatic cancer have high levels of anti-alternate reading frame protein antibodies. In another embodiment, patients that are actively suppressing HCV have a high anti-alternate reading frame protein positivity rate. In one embodiment, levels of antibodies to alternate reading frame polypeptides decline after the virus is cleared.

In one embodiment, to detect a particular clinical state (e.g., acute infection, chronic infection, liver cancer, response to treatment) an HCV polypeptide of any length, provided it immunoreacts with an antibody that specifically binds to a polypeptide comprising an amino acid sequence encoded by an HCV alternate reading frame relative to an amino acid sequence encoded by a standard HCV alternate reading frame. For example, in one embodiment, a polypeptide of the invention comprises at least about 8 amino acids, at least about 14 amino acids, at least about 18 amino acids, at least about 25 amino acids, at least about 50 amino acids, at least about 70 amino acids, or at least about 100 amino acids in length. In one embodiment, polypeptides comprising an amino acid sequence encoded by an HCV alternate reading frame described elsewhere (e.g., U.S. patent application 2002/0076415A1; Varaklioti et al. 2002. J. Biol. Chem. 277:17713) can be used.

In yet another aspect, the present invention provides an assay kit for diagnosing HCV infection in a subject. Preferably, the kit contains a solid support (e.g., an ELISA plate) and an HCV polypeptide of the invention. In one embodiment, a kit can comprise more than one polypeptide. In another embodiment, the solid support can be omitted from the kit. In another embodiment, the kit contains a solid support (e.g., an ELISA plate or a slide) and a monoclonal antibody or fragment thereof specific for a HCV alternate reading frame polypeptide. In one embodiment, a kit can comprise more than one antibody. In other embodiments, the solid support can be omitted. The assay kit can optionally include instructions, or additional reagents such as a solution for washing unbound proteins from the solid support, and materials needed for performing a detection assay.

C. Targets for Therapeutic Intervention

The polypeptides comprising an amino acid sequence encoded by an HCV alternate reading frame of the invention and/or nucleic acid molecules encoding them are also targets for anti-HCV therapy. As such the invention provides methods for identifying compounds which interact with a HCV alternate reading frame polypeptide and, thus, are likely to interfere with infection. In one embodiment, the method involves contacting the polypeptide with a compound in a cell-free system under conditions which allow interaction of the compound with the polypeptide such that a complex is formed. The complexes of polypeptide and compound can then be separated from the compounds which do not bind to the HCV polypeptide, the compounds which bind to HCV polypeptides can then be isolated and identified.

Exemplary compounds which can be screened for activity in the subject assays include, but are not limited to, peptides, nucleic acids, carbohydrates, small organic molecules, and natural product extract libraries. The term “non-peptidic compound” is intended to encompass compounds that are comprised, at least in part, of molecular structures different from naturally-occurring L-amino acid residues linked by natural peptide bonds. However, “non-peptidic compounds” are intended to include compounds composed, in whole or in part, of peptidomimetic structures, such as D-amino acids, non-naturally-occurring L-amino acids, modified peptide backbones and the like, as well as compounds that are composed, in whole or in part, of molecular structures unrelated to naturally-occurring L-amino acid residues linked by natural peptide bonds. “Non-peptidic compounds” also are intended to include natural products.

A recent trend in medicinal chemistry includes the production of mixtures of compounds, referred to as libraries. While the use of libraries of peptides is well established in the art, new techniques have been developed which have allowed the production of mixtures of other compounds, such as benzodiazepines (Bunin et al. 1992. J. Am. Chem. Soc. 114:10987; DeWitt et al. 1993. Proc. Natl. Acad. Sci. USA 90:6909) peptoids (Zuckermann. 1994. J. Med. Chem. 37:2678) oligocarbamates (Cho et al. 1993. Science. 261:1303), and hydantoins (DeWitt et al. supra). Rebek et al. have described an approach for the synthesis of molecular libraries of small organic molecules with a diversity of 104-105 (Carell et al. 1994. Angew. Chem. Int. Ed. Engl. 33:2059; Carell et al. Angew. Chem. Int. Ed. Engl. 1994. 33:2061).

The compounds of the present invention can be obtained using any of the numerous approaches in combinatorial library methods known in the art, including: biological libraries; spatially addressable parallel solid phase or solution phase libraries, synthetic library methods requiring deconvolution, the ‘one-bead one-compound’ library method, and synthetic library methods using affinity chromatography selection. The biological library approach is limited to peptide libraries, while the other four approaches are applicable to peptide, non-peptide oligomer or small molecule libraries of compounds (Lam, K. S. Anticancer Drug Des. 1997. 12:145).

In one embodiment, the test compound is a peptide or peptidomimetic. In another, preferred embodiment, the compounds are small, organic non-peptidic compounds.

Other exemplary methods for the synthesis of molecular libraries can be found in the art, for example in: Erb et al. 1994. Proc. Natl. Acad. Sci. USA 91:11422; Horwell et al. 1996 Immunopharmacology 33:68; and in Gallop et al. 1994. J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310); (Ladner supra.).

In many drug screening programs which test libraries of compounds and natural extracts, high throughput assays are desirable in order to maximize the number of compounds surveyed in a given period of time. Assays which are performed in cell-free systems, such as may be derived with purified or semi-purified proteins, are often preferred as “primary” screens in that they can be generated to permit rapid development and relatively easy detection of an alteration in a molecular target which is mediated by a test compound. Accordingly, in an exemplary screening assay of the present invention, the compound of interest is contacted with a HCV alternate reading frame polypeptide. Detection and quantification of HCV alternate reading frame polypeptide/compound complexes identifies the compound as a potential modulator of a HCV alternate reading frame polypeptide. The efficacy of the compound can be assessed by generating dose response curves from data obtained using various concentrations of the test compound. Moreover, a control, e.g., using a different polypeptide can also be performed to provide a baseline for comparison.

Complex formation between the HCV alternate reading frame polypeptide and a compound may be detected by a variety of techniques. For instance, modulation of the formation of complexes can be quantitated using, for example, detectably labelled proteins such as radiolabelled, fluorescently labelled, or enzymatically labelled HCV alternate reading frame polypeptides, by immunoassay, or by chromatographic detection.

Typically, it will be desirable to immobilize either the HCV alternate reading frame polypeptide or the compound or both to facilitate separation of compound/HCV alternate reading frame complexes from uncomplexed forms, as well as to accommodate automation of the assay. In one embodiment, a fusion protein can be provided which adds a domain that allows the polypeptide to be bound to a matrix. For example, glutathione-S-transferase/receptor (GST/receptor) fusion protein forms of the novel polypeptides can be adsorbed onto glutathione sepharose beads (Sigma Chemical, St. Louis, Mo.) or glutathione derivatized microtitre plates, which are then combined with the test compound which is bound to beads and incubated under conditions conducive to complex formation, e.g. at physiological conditions for salt and pH, though slightly more stringent conditions may be desired, e.g., at 4° C. in a buffer containing 0.6M NaCl or a detergent such as 0.1% Triton X-100. Following incubation, the uncomplexed forms are removed by washing and compounds which bind to polypeptides comprising an amino acid sequence encoded by an HCV alternate reading frame are identified.

Other techniques for immobilizing proteins on matrices are also available for use in the subject assay. For instance, the polypeptides comprising an amino acid sequence encoded by an HCV alternate reading frame can be immobilized utilizing conjugation of biotin and streptavidin. For instance, biotinylated polypeptides comprising an amino acid sequence encoded by an HCV alternate reading frame can be prepared from biotin-NHS(N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with the polypeptides comprising an amino acid sequence encoded by an HCV alternate reading frame can be derivatized to the wells of the plate, and the polypeptides comprising an amino acid sequence encoded by an HCV alternate reading frame trapped in the wells by antibody conjugation. As above, preparations of a HCV alternate reading frame polypeptide and a test compound are incubated in the wells of the plate, and the amount of polypeptides comprising an amino acid sequence encoded by an HCV alternate reading frame/compound complex trapped in the well can be quantitated.

Other exemplary methods for detecting such complexes, in addition to those described above for the GST-immobilized complexes, include immunodetection of complexes using antibodies reactive with the HCV alternate reading frame polypeptide, or which are reactive with the receptor protein and compete for binding with the HCV alternate reading frame polypeptide; as well as enzyme-linked assays which rely on detecting an enzymatic activity associated with the HCV alternate reading frame polypeptide. In the instance of the latter, the enzyme can be chemically conjugated or provided as a fusion protein with the HCV alternate reading frame polypeptide. To illustrate, the HCV alternate reading frame polypeptide can be chemically cross-linked or genetically fused with alkaline phosphatase, and the amount of HCV alternate reading frame polypeptide trapped in the complex can be assessed with a chromogenic substrate of the enzyme, e.g. paranitrophenylphosphate. Likewise, a fusion protein comprising the HCV alternate reading frame polypeptide and glutathione-S-transferase can be provided, and complex formation quantitated by detecting the GST activity using 1-chloro-2,4-dinitrobenzene (Habig et al (1974) J Biol Chem 249:7130).

For processes which rely on immunodetection for quantitating one of the proteins trapped in the complex, antibodies against the protein, such as the anti-HCV alternate reading frame antibodies described herein, can be used. Alternatively, the protein to be detected in the complex can be “epitope tagged” in the form of a fusion protein which includes, in addition to the HCV alternate reading frame polypeptide, a second polypeptide for which antibodies are readily available (e.g. from commercial sources). For instance, the GST fusion proteins described above can also be used for quantification of binding using antibodies against the GST moiety. Other useful epitope tags include myc-epitopes (e.g., see Ellison et al. (1991) J Biol Chem 266:21150-21157) which includes a 10-residue sequence from c-myc, as well as the pFLAG system (International Biotechnologies, Inc.) or the pEZZ-protein A system (Pharamacia, N.J.).

VI. Nucleic Acid Structures Involved in Production of Alternate Reading Frame Polypeptides

As an alternative to, or in addition to, detection of alternate reading frame polypeptides, nucleic acid molecules encoding those polypeptides and/or nucleic acid signals and/or structures involved in the production of alternate reading frame polypeptides can be assayed to determine whether a patient is producing alternate reading frame polypeptides or is likely to produce such polypeptides. In another embodiment, these nucleic acid sequences can be used as targets for therapeutic intervention.

In one embodiment, the invention pertains to a method of predicting the propensity of a patient infected with an HCV virus to clear HCV spontaneously, to clear HCV in response to IFN, to progress to chronic infection, to fail IFN treatment, to develop chronic HCV disease, cirrhosis, or liver cancer comprising detecting sequence changes in HCV RNA molecules.

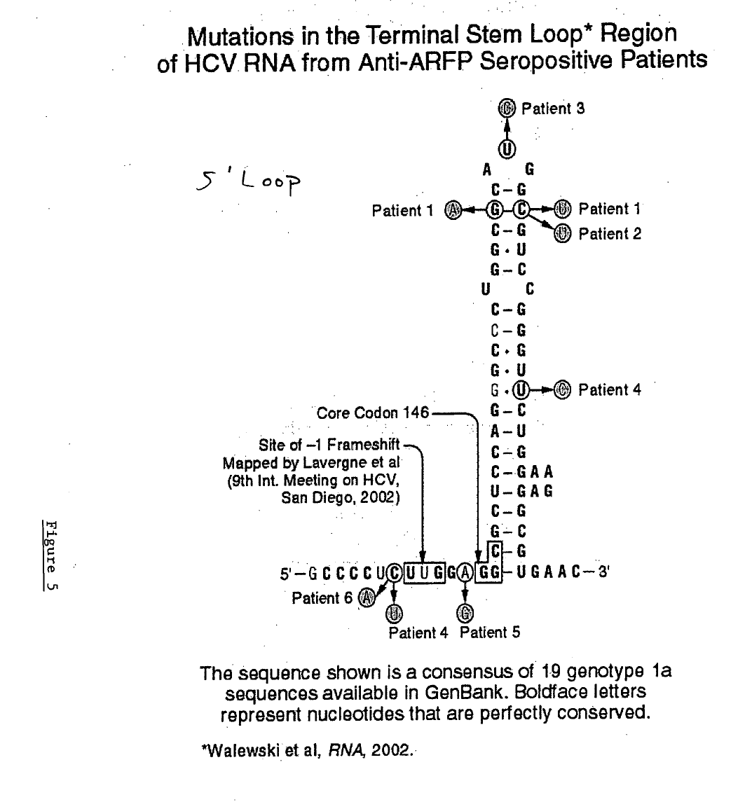

In one embodiment, the virus population present in a patient can be sampled, e.g., by cloning viral nucleic acid molecules from the patient and sequencing them using standard techniques. In one embodiment, the sequence of the viral nucleic acid molecules can be used to determine the potential of the patient to produce an alternate reading frame protein. For instance, in one embodiment, the 5′ and/or 3′ Stem-loops in the Terminal Stem-loop region can be sequenced and the sequences examined for mutations (FIG. 1 and FIG. 5). The Terminal Stem-loop region is near the 3′ end of the core polyprotein encoding region (Walewski et al. 2001. RNA 7:710.; Walewski et al. 2002 RNA 8:557; Ogata et al. 2002. J. Clin. Microbiol. 40:3625). Mutations in the Terminal Stem-loops accumulate during progression to advanced liver disease and liver cancer, thereby modulating the function of cis regulatory elements and increasing production of alternate reading frame polypeptides leading to expression of anti-alternate reading frame polypeptide antibodies. Mutations in this region can interfere with the structure of one or both of the stem loops. For example, mutations can disrupting a stem loop, e.g., by altering the hydrogen bonding character of the stem loop (e.g., by disrupting the binding of two nucleotides or by mutating from a G-C binding pair to an A-T binding pair within the stem loop and, thereby reducing the number of hydrogen bonds). Mutations can also create and/or interfere with recognition of a frameshift site (e.g., as described by Layergne et al. supra) or can introduce a stop codon (e.g., prior to the 5′ stem loop). Mutations in the Terminal Stem loops region favor production of alternate reading frame polypeptides. Accordingly, the presence of mutation in this region can be used as an indicator that a virus or viral population is producing or is likely to produce alternate reading frame polypeptides. In another embodiment, the detection of mutations in other RNA elements that modulate ARFP expression is detected.

In one embodiment, a reporter system can be employed for functional analysis of the terminal stem loops. For example, in one embodiment, a reporter construct comprises a, from 5′ to 3′, a T7 promoter, an alpha-globin mRNA leader sequence, 6 codons of globin mRNA, an HSV nucleic acid molecule comprising stem-loop 1 and stem-loop 2, a signal peptide coding sequence, and a reporter protein. The reporter protein sequence comprises a stop codon in each of the three reading frames. The stop codons are spaced so that the products of each reading frame can be distinguished by size on a gel. Using such a system, different HSV nucleic acid molecules can be tested for their affect on ARFP production (e.g., molecules from different patient populations) or the effect of test compounds on ARFP production can be tested.

In another embodiment, an in vitro translation assay can be performed to translate a viral nucleic acid molecule or a transcript representing this molecule in vitro using standard techniques. The product of the in vitro translation can be tested (e.g., using SDS page, Western blotting, or ELISA) to determine whether an alternate reading frame protein is being made by the virus.

The practice of the present invention will employ, unless otherwise indicated, conventional techniques of cell biology, cell culture, molecular biology, microbiology, recombinant DNA, and immunology, which are within the skill of the art. Such techniques are explained fully in the literature. See, for example, Genetics; Molecular Cloning A Laboratory Manual, 2nd Ed., ed. by Sambrook, J. et al. (Cold Spring Harbor Laboratory Press (1989)); Short Protocols in Molecular Biology, 3rd Ed., ed. by Ausubel, F. et al. (Wiley, NY (1995)); DNA Cloning, Volumes I and II (D. N. Glover ed., 1985); Oligonucleotide Synthesis (M. J. Gait ed. (1984)); Mullis et al. U.S. Pat. No. 4,683,195; Nucleic Acid Hybridization (B. D. Hames & S. J. Higgins eds. (1984)); the treatise, Methods In Enzymology (Academic Press, Inc., N.Y.); Immunochemical Methods In Cell And Molecular Biology (Mayer and Walker, eds., Academic Press, London (1987)); Handbook Of Experimental Immunology, Volumes I-IV (D. M. Weir and C. C. Blackwell, eds. (1986)); and Miller, J. Experiments in Molecular Genetics (Cold Spring Harbor Press, Cold Spring Harbor, N.Y. (1972)).

The contents of all references, pending patent applications and published patents, cited throughout this application are hereby expressly incorporated by reference.

The invention is further illustrated by the following examples, which should not be construed as further limiting.

### EXAMPLES

### Example 1

### The Detection of Immune Responses Against Polypeptides Comprising an Amino Acid Sequence Encoded by an HCV Alternate Reading Frame

Consensus polypeptides were synthesized based on the sequence homology between polypeptides comprising an amino acid sequence encoded by an HCV alternate reading frame shown in Table 1. The following peptides were made using conventional techniques by Biosynthesis Corp. (Lewisville, Tex.): LNLKEKPNVTPTAC and AAHRTSSSRAVVRC (alternate reading frame (ARF) polypeptides 1 and 2, respectively). The following polypeptides derived from HCV CORE protein were used as controls: PTDPRRRSRNLGKVIDTC and GCATRKTSERSQPRGRRAPI. The peptides were conjugated to activated bovine serum albumin giving up to 4 peptide molecules for each BSA molecule. Peptide-BSA conjugates at a concentration of approximately 0.5 mg/ml were shipped on ice in 3 mls of phosphate buffered saline, 0.1 M, pH 7.4. They were aliquoted and stored at −20° C. until use.

ELISAs were performed as described by Kirkegaard and Perry Laboratories, Inc. (Gaithersburg, Md.). For use in the ELISA, the polypeptides were dissolved in 1× “coating buffer” to give a peptide-BSA concentration of 10 μg/ml; 100 μl were added to 96 well microtiter plates (Falcon 3072, Becton Dickinson and Company, Franklin Lakes, N.J.) and incubated at room temperature for 1 hour or overnight at 4° C. The coating solution was removed and plates were then blocked by adding 300 μl of 1% BSA in phosphate buffered saline (as prepared by Kirkegaard and Perry), and incubating for 30 min. at room temperature. The blocking solution was removed and 100 μl of 1% BSA (1×) was added.

Sera was obtained from patients which were known to have or to have previously had an HCV infection. Serum samples were diluted 1:100 in 1×BSA and 100 μl was added to the first well of each (yielding a total of 200 μl). Serum samples were then serially diluted (two-fold at each step). Control wells contained BSA only. Plates were incubated for 1 hour at room temperature with moderate agitation to allow binding. Plates were washed 5 times with 1× wash solution (PBS and 0.02% Tween). After washing, 100 μl of the secondary antibody was immediately added and allowed to react for 1 hour at room temperature. The secondary antibody was either the Fab fragment of anti-human IgG conjugated to horse radish peroxidase (HRP) at a dilution of 1:1000, or anti-human IgG (in PBS with 1% BSA). Plates were washed 5 times with 1× washing solution, and then 100 μl of hydrogen peroxide and TMB were added and allowed to react for 10 to 30 minutes at room temperature. To stop the reaction, 100 μl of 1M phosphoric acid were added. O.D. measurements were obtained by using a dual wavelength scanner: 450 nm values-650 nm values.

Results of ELISA Tests for Antibodies of HCV-Specific Polypeptides

| | Control Sera | HCV Patient Sera |
|---|---|---|
| | Mean OD (S.D.) | Mean OD (S.D.) |
| | (N = 2, tested | (N = 6, tested |
| | in duplicate) | in duplicate) |
| CORE #1 | 0.379 (0.063) | 0.851 (0.231) |
| CORE #2 | 0.775 (0.053) | 1.390 (0.400) |
| ARF #1 | 0.763 (0.133) | 2.286 (0.215) |
| ARF #2 | 0.401 (0.039) | 0.813 (0.813) |

Alternate reading frame polypeptides were also found to detect T cell responses. Peripheral blood mononuclear cells were prepared from 9 HCV patients and controls. The stimulation index was calculated as the percentage of proliferating CD3+ cells in cultures incubated with an ARFP peptide divided by the percentage of proliferating cells in cultures incubated with an irrelevant peptide. The cells of 9 HCV patients proliferated when cultured for 6 days in the presence of 25 ug/ml ARFP peptide and also when cultured for 9 days with either 5 or 25 ug/ml of ARFP peptide.

### Example 2

### Development of a Western Blot Assay for Detecting Antibody Production to Alternate HCV Reading Frame Proteins

Five micrograms of BSA-alternate reading frame peptide conjugates and appropriate contols (quenched BSA, BSA-BSA peptide conjugates) were denatured by incubating in standard Lamelli loading buffer (with beta-mercaptoethanol and SDS), at 100° C. for three minutes. Samples were then cooled and spun in a microcentrifuge prior to loading on a discontinuous 1.5 mm thick SDS-PAGE gel (4% stacking gel [pH 6.8] and 12.5% [pH 8.4] resolving gel). The running buffer was TRIS-glycine/SDS (0.1%), with an unadjusted pH of approximately 8.4.

Samples were run through the stacking gel (1 cm) for approximately 45 mins at 90 volts. After the bromophenol blue (BB) entered the resolving gel, the voltage was increased to 160 V. The gels were run for approximately 1.5 hrs, or until the BB was ⅔ of the way through the gel. After stopping the run, the gels were either stained with coomassie blue, or equlibrated for 15 minutes with the transfer buffer (1×CAPS, pH 11.0, 10% MeOH. PVDF membranes (Immobilon-P [0.45 micon pore size], were wet in 100% MeOH for 1 min, then 50:50 MeOH: 18MΩ water for 5 mins, and finally equilibrated with the transfer buffer. The transfer was set up in transfer buffer (filter paper, gel, PVDF membrane, and filter paper), and slipped into the BIO-RAD transfer tank. The transfer was run at 20 volts, overnight (approximately 10 hrs), at 4° C., with stirring.

After transfer, the tank was disassembled, the membrane was rinsed in 18MΩ water for 1 minute, then soaked in 100% MeOH for one minute, then allowed to air-dry. In order to visualize the efficiency of the transfer, the membranes (after re-wetting in MeOH then 18MΩ water) were incubated with 1% Ponceau-S red stain and rinsed in 18MΩ water After scanning the image, the dye was removed in a dilute NaOH solution (1 ml saturated NaOH in 100 mls 18MΩ water.

The membranes were then blocked in 3% NFDM (6 grams non-fat dried milk in 200 mls of 1×TBS [TRIS-buffered saline, ph 7.4), for one hour. After blocking, the membranes were rinsed in two washes of 1×TBS, 200 mls apiece.

The membranes were then incubated with the primary antibody solution 4 mls of a 1/200 dilution of patient sera in 1% BSA in 1×TTBS (TBS with 0.025% Tween-20). This incubation lasted one hour, at 30° C., in glass with slow rotation of the tube or beaker. After this incubation, the membranes were washed three times in 200 mls of 1×TTBS.

The secondary antibody solution was 200 mls of a 1/3000 dilution of the BIO-RAD goat anti-human alkaline phosphatase conjugate, in 1% BSA in 1×TTBS. This incubation lasted 1 hr, at 30° C., with gentle shaking.

After this incubation, the membranes were washed twice with 200 ml of 1×TTBS (5 mins apiece), and one with 200 mls of 1×TBS.

The bands were visualized with the BIO-RAD AP substrate (200 mls total), with gentle, occasional shaking. After visualization, the membranes were washed several times with 18MΩ water, followed by one wash with MeOH, then air-dried and photographed or scanned.

### Example 3

### Preparation of Long Synthetic Alternate Reading Frame Polypeptides

A consensus polypeptide (100 amino acids in length) representing the “approximate middle” of the HCV alternate reading frame protein were synthesized by QCB (Hopkinton, Mass.). This peptide contains ARFP amino acids ˜41-140 and has the sequence of a genotype 1a consensus:

GALDWVCARRGRLPSGRNLEVDVSLSPRHVGPRAGPGLSPGTLGPSMAM
RAAGGRDGSCLPVALGLAGAPQTPGVGRAIWVRSSIPLRAASPTSWGTY
RS.

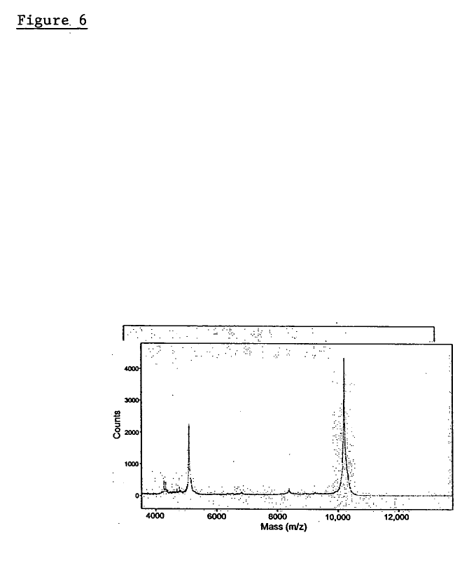

Mass spectroscopy confirmed that the polypeptide has the expected molecular weight, 10.5 kD (FIG. 6). The peptide preparation is over 90% pure full-length peptide, as indicated by HPLC.

The peptide was synthesized by conventional chemistry, purified by HPLC and Mass spec, lyophilized and shipped overnight at 4° C. The lyophilized ARFP 100mer peptide was stored at −20° C., until dissolved in a 1% glacial acetic acid solution at a concentration of 1 mg/ml and frozen. The free amide form of the polypeptide was used in ELISA assays or conjugated to biotin before use.

### Example 4

### Long Synthetic Alternate Reading Frame Polypeptides React with Patient Sera in Western Blots

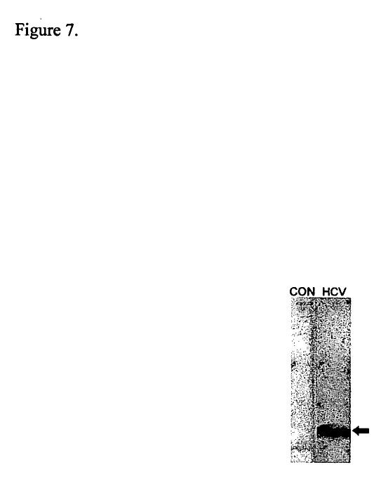

Western blotting reveals that the new alternate reading frame polypeptide reacts with serum from an HCV patient, but not from a control (FIG. 7).

### Example 5

### Long Synthetic Alternate Reading Frame Polypeptides React with Patient Sera in Elisa Assays

For coating the ELISA plates, the polypeptide was diluted to a final concentration of 10 μg/ml (the final concentration can range from 0.1 to 100 ug/ml as an average, in 1× coating buffer (KPL, Gaithersberg, Md.). 100 ul of this coating solution was added to each well of a MAXIsorb microwell plate (NUNC, Denmark) and incubated overnight at 4° C. This assay can also be performed on a number of NUNC plates with different surface chemistries, such as Multisorp, Medisorp, and Immobilizer Amino plates. The latter plate has the advantage of a surface chemistry which will covalently link peptides by their amino terminus to the plate when these are incubated for an hour at room temperate in 1×PBS, pH7.4. The concentration of the peptides in the coating buffer for this application can range from 0.1 to 10 μgm/ml 1×PBS, pH 7.4.

When the biotin-conjugate form of the 100mer peptide was used, the plate coating procedure was altered. Biotin conjugated polypeptide was rehydrated in 1% glacial acetic acid, and diluted to a final concentration of 10 μg/ml in 1×PBS, pH 7.0, tween-20 (0.05%). 100 μl/well of this coating solution were added to pre-rinsed [3×330 μl/well 1×PBS, pH 7.0, tween-20 (0.05%)] strepavidin-coated microwell plates (Reacti-bind plates, Pierce Biotechnology, Rockford, Ill.; or, BD Biocoat Strepavidin Assay Plates, BD Biosciences, Bedford, Mass.).

After warming the plates to “room temperature”, the coating solution was recovered and stored at −20° C. The plates then blocked with 5% Non-Fat Dried Milk (Bio-Rad, Hercules, Calif.), in 1×PBS, with 0.05% Tween-20 (Sigma, St. Louis, Mo.), for one hour at 30° C., by adding 330 μl/well. The blocking solution was then discarded, and the wells were rinsed with 330 μl/well of “wash buffer” (1×PBS, with 0.05% Tween-20, and 0.1% BSA).

Sera was obtained from patients which were known to have or to have previously had an HCV infection. As negative controls, patients known to have other infectious hepatitis (HBV), or other non-infectious liver disease (hemochromatosis, PBC) were included in the assay. Also, serum samples from patients with no known liver diseases were included. Serum samples were initially diluted 1/400 in 2% BSA in 1×PBS with 0.05% Tween-20. These initial dilutions were further diluted to a final concentration of 1/4000, by adding 22 μl of the 1/400 dilution, to 198 μl of 2% BSA in 1×PBS with 0.05% Tween-20. 100 μl of the final dilution was added to the appropriate well of pre-blocked plates described above. Blank wells received the 2% BSA only (no primary antibody). Plates were incubated for 1 hour at 30° C., with gentle rotation (150 RPM), to allow binding of the patient antibodies to the ARFP peptide. The plates were then washed 5 times (5 minutes per wash), with 330 μl/well of “wash buffer” (1×PBS, with 0.05% Tween-20, and 0.1% BSA).

After the 5^{th} wash the secondary antibody (goat-anti human IgG with HRP conjugate (Jackson Labs) (1/20,000) in 2% BSA in 1×PBS (pH 7.0) with 0.05% Tween-20 was added (100 μl/well). were incubated for 45 minutes at 30° C., with gentle rotation (150 RPM), to allow binding of the secondary antibody. The plates were then washed 5 times (5 minutes per wash), with 330 μl/well of “wash buffer” (1×PBS, with 0.05% Tween-20, and 0.1% BSA).

After the second wash series, 100 μl/well of TMB substrate and hydrogen peroxide were added per well for 5 to 30 minutes at room temperature. The reaction was stopped with 100 μl/well of a 10% Phosphoric Acid solution. O.D. measurements were obtained with a dual wavelength scanner: O.D.=abs₄₅₀−abs₆₅₀.

Results for anti-ARFP antibodies using the consensus 1a 100mer peptide (10 μg/ml) on MAXIsorb plates (NUNC) are shown below. Each sample was assayed in duplicate; the values below represent the means of the duplicates. OD values represent the absorbance at 450—the background at 650. The mean for 7 negative controls was 0.027, with a standard deviation of 0.0327. The threshold for “positivity” was 0.125. Responses above the threshold are in bold.

| | | OD₄₅₀-OD₆₅₀ | SD | X + 3SD |
|---|---|---|---|---|
| Controls | | X = 0.027 | 0.0327 | 0.125 |
| HCC/HCV | 1 | 0.095 | | |
| | 2 | 0.140 | | |
| | 3 | 1.180 | | |
| | 4 | 0.134 | | |
| | 5 | 0.042 | | |
| | 6 | 0.060 | | |
| | 7 | 0.046 | | |
| | 8 | 0.016 | | |
| | 9 | 0.023 | | |
| | 10 | 0.195 | | |
| | 11 | 0.043 | | |
| | 12 | 0.140 | | |
| | 13 | 0.035 | | |
| | 14 | 0.018 | | |
| | 15 | 0.035 | | |
| | 16 | 0.046 | | |
| | 17 | 0.018 | | |
| HCC/HBV | 1 | 0.000 | | |
| | 2 | 0.000 | | |
| | 3 | 0.047 | | |
| | 4 | 0.005 | | |
| | 5 | 0.027 | | |
| | 6 | 0.030 | | |
| | 7 | 0.002 | | |
| | 8 | 0.024 | | |
| | 9 | 0.030 | | |
| | 10 | 0.015 | | |
| | 11 | 0.000 | | |
| | 12 | 0.002 | | |
| | 13 | 0.015 | | |
| | 14 | 0.000 | | |
| | 15 | 0.000 | | |

The assay cut off as the mean of the healthy controls plus 3 times the SD. 5 out of 17 patients were positive, with an overall rate of 30% in the HCC/HCV, versus 0% for the HCC/HBV population. A similar assay showed 53% reactivity in the HCC/HCV group while none of the HBV/HCC patients gave a signal above the cut off value. HBV/HCC patients were used as the comparison group in this study to rule out the possibility that HCC (hepatocellular carcinoma) itself is associated with anti-ARFP antibodies. Initial results suggest that about 10% of an unselected group of chronically infected HCV patients without HCC have anti-ARFP antibodies against this peptide. This value is about twice that obtained with the previous ELISA, showing the utility of long peptides. This initial experiment indicates that alternate reading frame polypeptides are produced at a significant level and stimulate antibody responses in about 50% of chronically infected HCV patients with liver cancer.

Additional ELISA assay development using the 100mer peptide described above to coat new microwell plates from NUINC confirmed the initial findings. In these assays, 19 of 72 HCV positive patients reacted with the 100mer, for an overall rate of 26% positivity. An additional 3 patients of this group of 72 were positive for anti-ARFP antibodies when the antigen in the indirect ELISA assay was the 70mer peptide described in this application. The combined reactivity rate for either of the two peptides was 22 of 72 HCV positive patients, for an overall rate of 31% for patients with anti-ARFP antibodies.

### Example 6

### Correlation of Alternate Reading Frame Polypeptides with Cell Proliferation

In addition, anti-alternate reading frame antibody levels have been found to rise upon treatment with IFN. This indicates that IFN treatment stimulates production of Alternate reading frame polypeptides. Alternate reading frame polypeptide may promote viral replication by promoting cellular proliferation. Consistent with this finding is the fact that plasmids bearing the core/alternate reading frame protein gene promote cellular transformation (Ray et al. 1996. J. Virol). In addition, the core/alternate reading frame polypeptide genes of many HCC patients overexpress alternate reading frame polypeptides (Yeh et al. 2000 J. Gastro and Hepatology).

### Example 7

### Expression of ARFP in HEK 293T Cells

The ARFP protein sequence was submitted to GENEART GmbH (Regensburg, Germany) for the production of a nucleotide sequence which was codon optimized for expression in Homo sapiens and other mammals.

The codon usage was adapted to the codon bias of Homo sapiens genes. In addition, regions of very high (>80%) or very low (<30%) GC content were avoided where possible. During the optimization process the following cis-acting sequence motifs were avoided:
internal TATA-boxes, chi-sites and ribosomal entry sites
AT-rich or GC-rich sequence stretches
repeat sequences and RNA secondary structures
(cryptic) splice donor and acceptor sites, branch points

This nucleotide sequence was designed in order to express ARFP protein with little to no frameshifting. The codon optimized ARFP sequence is:

GCCACCATGGCCAGAATCCTGAACCTGAAGAAGAAAACCAACGTGACCCC
CACCGTGGCCCACAGGACCAGCAGCAGCAGAGTGGCCGTGAGGAGCCTGG
TGGAGTTCACCTGCTGCAGGGCCGGAGCCCTGGACTGGGTGTGCGCCAGG
AGGGAGAGGCTGCCCAGCGGCAGGAACCTGGAAGTGGACGTGTCTCTGAG
CCCCAGACTGGTGGGCCCCAGAGCCGGACCCGGCCTGTCCCCTGGCACCC
TGGGCCCTAGCATGGCTATGAGGGCCGCAGGCGGAAGGGACGGCAGCTGT
CTGCCCGTGGCACTGGGCCTGGCTGGCGCCCCTCAGACACCTGGCGTGGG
CAGAGCCATCTGGGTGAGAAGCAGCATCCCCCTGAGAGCCGCCTCCCCTA
CCTCTTGGGGCACCTACAGGAGCAGCGCCCCACTGCTGGAAGCCCTGCCT
GGCCCTTGGAGGATGGCCTCTGGCTTCTGGAAAACCGCCTGATGA

This ORF found in the above nucleotide sequence encodes a polypeptide of approximately 161 AA, which is presented below:

MARILNLKKKTNVTPTVAHRTSSSRVAVRSLVEFTCCRAGALDWVCARRE
RLPSGRNLEVDVSLSPRLVGPRAGPGLSPGTLGPSMAMRAAGGRDGSCLP
VALGLAGAPQTPGVGRAIWVRSSIPLRAASPTSWGTYRSSAPLLEALPGP
WRMASGFWKTA

GENEART assembled the sequence from overlapping oligonucleotides, cloned the sequence into the multiple cloning site of pPCR-script for sequencing and amplification. Once the sequence was completely verified, 10 μl of the plasmid prep was lyophilized.

The plasmid was transfected into bacteria for amplification and sequence verification. Once the expected sequence was verified, the ARFP encoding insert was transferred into a mammalian expression vector of the Vitality® hrGFP II series from (Stratagene, La Jolla, Calif.). The optimized ARFP sequence was cloned into the vector multiple cloning site after a double digest of the expression vector pIRES-hrGFP II with EcoR I and Not I. After standard protocols to select and screen the new construct, it was confirmed by direct sequencing. For transient transfection of the plasmid (15 μgms/10 cm dish) with FuGENE 6 (ROCHE, Indianapolis, Ind.), the cells were plated onto 10 cm tissue culture dishes. The cells were cultured under standard conditions to approximately 80% confluence.

Since initial expression of the ARFP was poor with this construct, a derivative construct was designed where the IRES-GFP encoding region was removed, since it was hypothesized that this region might interfere with the expression of the ARFP encoding region. Transient transfections with this construct (pARF-ΔIGFP) were performed according to the above procedure, with positive results.

Western Blot Analysis of ARFP Expressing Cells.

Extracts of the cells were prepared 2 days pot-transfection after washing the mono-layers 2× with 1×PBS. 0.7 mls of lysis buffer (10 mM TRIS pH7.2, 150 mM NaCl, 1% TX-100 containing protease inhibitor cocktail [Complete EDTA-free (Roche Diagnostics, GmBH) were used to harvest the cells. The extracts were sonicated, then centrifuged (13 KG, 10 mins) to precipitate insoluble materials.

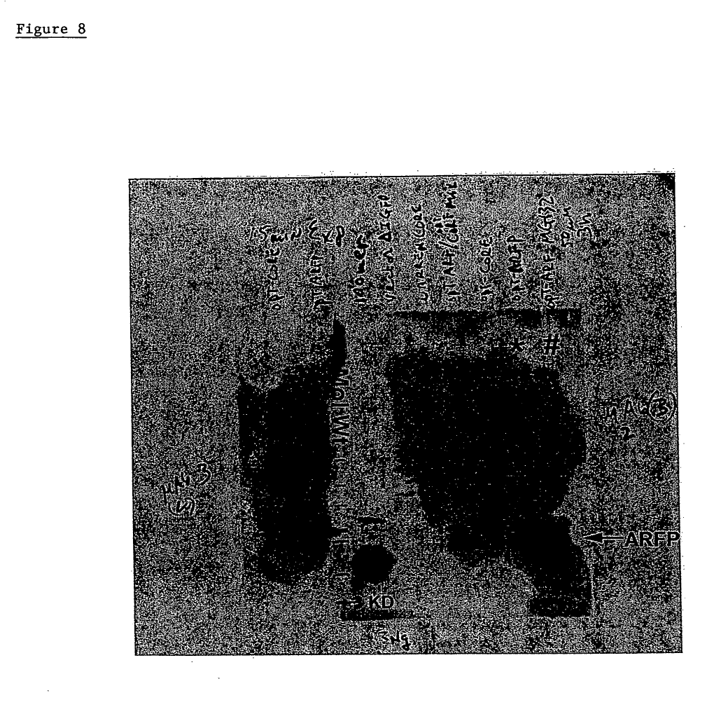

Soluble fractions were separated by SDS-PAGE using 15% NuPAGE gels (Invitrogen) according to the manufacturer's protocols. Proteins were transferred onto PVDF membranes, and the blots were probed with anti-ARFP mouse monoclonals that are directed against the 100mer peptide. A goat-anti-mouse HRP conjugate with chemiluminescent substrate was used to visualize the relevant bands (see FIG. 8). These studies clearly show that the pARF-ΔIGFP construct expressed a protein of the expected size of approximately 14.5 KD, which strongly reacts with two distinct monoclonal antibodies (numbers 2 and 3 which recognize different epitopes of ARFP).

### Example 8

### Panels of Alternate Reading Frame Polypeptides and Antibody Reactivity

Panels of peptides were derived from a consensus genotype 1a alternate reading frame amino acid sequence The peptides were designed with the C-terminal amino acids G, P, E, D, Q, N, T, S, and C being forbidden and the N-terminal amino acid Q being forbidden.

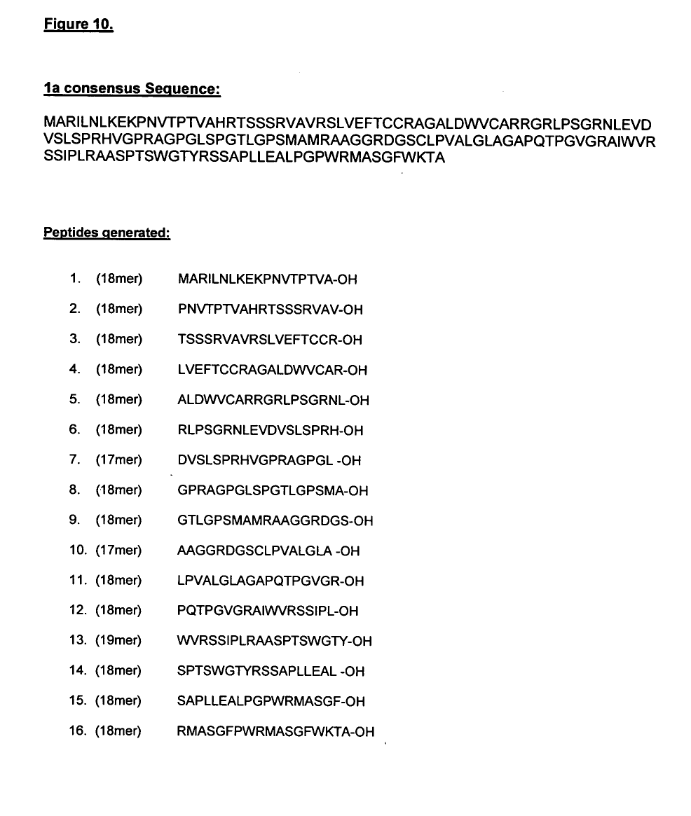

18mer peptides were designed to span the entire 1a consensus sequence and can be used, e.g., in standard ELISA assays to screen patient sera or to make an epitope map of the binding site of monoclonal antibodies to a narrow region of the ARFP sequence. Exemplary polypeptides are shown in FIG. 10.

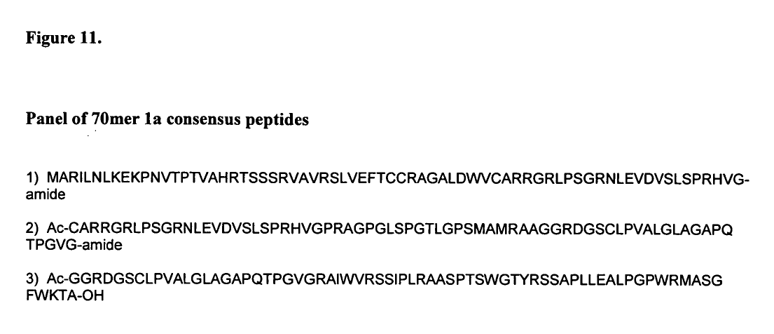

Longer 70mer peptides were also designed and exemplary polypeptides are shown in FIG. 11.

Monoclonal antibodies were made against a 100mer alternate reading frame polypeptide. Supernatants that were positive by ELISA assay were further screened by Western blot assay to test the utility of specific monoclonal. Those that passed this stage of screening were selected for further development by fusion to myeloma cells, and these were subjected to cloning and repeated screening, growth, and purification of the supernatant.

18mer peptides were tested for reactivity with five different monoclonal antibodies (m1-m5). The peptide from FIG. 10 with which each monoclonal specifically reacted is shown under each monoclonal (e.g., peptide 12, 11, etc.) The concentrations of the individual monoclonals in each assay are highlighted in green in the first column. Monoclonal 3 reacts with what appears to be an epitope that is present in both peptides 12 and 13, which is reasonable since these overlap by approximately 8 amino acids.

Table of Monoclonal Titration Series v. ARFP 18mer Containing
Specific Epitope

| | m1 | m2 | m3 (a) | m3 (b) | m4 | m5 |
|---|---|---|---|---|---|---|
| | Peptide | Peptide | Peptide | Peptide | Peptide | Peptide |
| Concentration | 12 | 11 | 12 | 13 | 10 | 8 |
| 0.005 | 0.161 | 0.322 | 0.15 | 0.01 | 0.013 | 0.018 |
| 0.01 | 0.248 | 0.477 | 0.213 | 0.013 | 0.013 | 0.02 |
| 0.02 | 0.399 | 0.871 | 0.398 | 0.013 | 0.019 | 0.047 |
| 0.04 | 0.593 | 1.182 | 0.676 | 0.022 | 0.026 | 0.06 |
| 0.08 | 0.828 | 1.422 | 0.951 | 0.029 | 0.033 | 0.132 |
| 0.156 | 1.181 | 1.559 | 1.187 | 0.053 | 0.082 | 0.228 |
| 0.3125 | 1.544 | 2.037 | 1.73 | 0.098 | 0.114 | 0.322 |
| 0.625 | 1.791 | 2.013 | 1.744 | 0.233 | 0.241 | 0.695 |
| 1.25 | 1.738 | 1.741 | 1.934 | 0.404 | 0.4 | 0.831 |
| 2.5 | 1.742 | 1.673 | 1.76 | 0.576 | 0.619 | 1.35 |
| 5 | 1.849 | 1.875 | 1.958 | 0.879 | 0.921 | 1.588 |
| 10 | 1.738 | 1.724 | 1.947 | 1.288 | 1.163 | 1.687 |

Monoclonal 3 reacts with what appears to be an epitope that is present in both peptides 12 and 13. This is not surprising as these overlap by approximately 8 amino acids. Values are OD readings by Indirect ELISA.

1. An isolated, synthetic, or recombinant polypeptide of at least about 100 amino acids in length, wherein the polypeptide is immunoreactive with antisera that specifically detects an HCV alternate reading frame polypeptide relative to an HCV standard reading frame polypeptide, and wherein the polypeptide comprises a consensus amino acid sequence.

2. The polypeptide of claim 1, the polypeptide comprises an amino acid sequence encoded by a reading frame +1 to the main HCV open reading frame.

3. The polypeptide of claim 1, wherein the polypeptide comprises the sequence:
GALDWVCARRGRLPSGRNLEVDVSLSPRHVGPRAGPGLSPGTLGPSMAM
RAAGGRDGSCLPVALGLAGAPQTPGVGRAIWVRSSIPLRAASPTSWGTY
RS.

4. The polypeptide of claim 1, wherein the polypeptide comprises the sequence:
GAPGWVCARLGRLPSGRNLVEGD(S/N)LSPRLAGPRAPGLSPGTLGPSM
AMRAWGGQDGSCPPAALGLVGAPRTPGVGRAIWVRSSIPSHAASPTSWGT
RS.

5. The polypeptide of claim 1, wherein the polypeptide comprises the sequence:
GRLPSGRSLVEGASLSPRIAGPRAGPCLSPGTLGPSMAMRVAGGQDGSCP
PAALGLLGAPMTPGGGPAIWVRSSIPLRAASPTSWGTSRS

6. The polypeptide of claim 1, wherein the polypeptide comprises an amino acid sequence that varies from the consensus amino acid sequence shown in FIG. 9 by substitution of between about one and about twenty amino acids.

7. The polypeptide of claim 1, which further comprises a non-HCV polypeptide or other material.

8. The polypeptide of claim 7, wherein the non-HCV polypeptide or other material increases an immune response to the polypeptide.

9. The polypeptide of claim 1, which further comprises an amino acid sequence encoded by the HCV standard reading frame.

10. A polypeptide comprising the amino acid sequence:
MARILNLKEKPNVTPTVAHRTSSSRVAVRSLVEFTCCRAGALDWVCARRG
RLPSGRNLEVDVSLSPRHVGPRAGPGLSPGTLGPSMAMRAAGGRDGSCLP
VALGLAGAPQTPGVGRAIWVRSSIPLRAASPTSWGTYRSSAPLLEAIPGP
WRJVIASGFWKTA

11. A composition comprising the polypeptide of claim 1 and a pharmaceutically acceptable carrier.

12. A method of preventing HCV infection comprising administering the composition of claim 11 to a subject.

13. A method of preventing chronic HCV infection comprising administering at least one isolated, synthetic, or recombinant polypeptide to a subject, wherein the polypeptide comprises an immunogenic portion of an HCV alternate reading frame polypeptide.

14. A method of preventing chronic HCV infection comprising administering the polypeptide of claim 11 to a subject.

15. A method of increasing clearance of HCV in a subject during an ongoing infection comprising administering at least one isolated, synthetic, or recombinant HCV polypeptide to the subject, wherein the polypeptide comprises an immunogenic portion of an HCV alternate reading frame polypeptide.

16. A method of increasing clearance of HCV comprising administering the polypeptide of claim 11 to a subject.

17. A method of predicting the response of a subject to a therapy for HCV comprising detecting the presence or absence of a polypeptide in the body fluid or cells of a subject, wherein the polypeptide is immunoreactive with antisera that specifically detects an HCV alternate reading frame polypeptide relative to an HCV standard reading frame polypeptide, or the presence or absence of antibodies which bind to the polypeptide, wherein the presence of the polypeptide or antibodies which bind to the polypeptide is predictive of a favorable response to therapy for HCV.

18. The method of claim 17, wherein the therapy comprises administration of IFN.

19. A method of predicting the propensity of a patient infected with an HCV virus to clear HCV spontaneously, to clear HCV in response to IFN, to progress to chronic infection, to fail IFN treatment, to develop chronic HCV disease, cirrhosis, or liver cancer comprising detecting sequence changes in the amino acid sequence of alternate reading frame polypeptides made by the virus or the RNA molecules encoding them.

20. The polypeptide of claim 1, which is conjugated to a moiety that facilitates detection or attachment to a matrix.

21. The polypeptide of claim 1, which is immobilized on a matrix.

22. A method of diagnosing acute HCV infection comprising detecting the presence or absence of a polypeptide comprising an amino acid sequence encoded by an HCV alternate reading frame or detecting the presence or absence of antibodies which bind to a polypeptide comprising an amino acid sequence encoded by an HCV alternate reading frame in the body fluid or cells of a subject, wherein the presence of the polypeptide or antibodies which bind to the polypeptide is indicative of acute infection with HCV.

23. A method of diagnosing acute HCV infection, comprising detecting the presence or absence of antibodies in the body fluid of a subject, wherein the antibodies react with a polypeptide comprising an amino acid sequence encoded by an HCV alternate reading frame or a consensus polypeptide comprising a consensus sequence based on HCV alternate reading frame proteins encoded by more than one viral genotype, and wherein the presence of the antibodies is indicative of an acute infection with HCV.

24. The method of claim 23, wherein the polypeptide is the polypeptide of claim 1

25. A method of diagnosing acute Hepatitis C virus (HCV) infection, comprising detecting the presence or absence of a polypeptide in the body fluid or cells of a subject, wherein the polypeptide comprises an amino acid sequence encoded by an HCV alternate reading frame, and wherein the presence of the polypeptide is indicative of acute infection with HCV.

26. The method of claim 25, wherein the polypeptide is the polypeptide of claim 1.

27. A method of diagnosing liver cancer or advanced cirrhosis comprising detecting the presence or absence of a polypeptide comprising an amino acid sequence encoded by an HCV alternate reading frame or detecting the presence or absence of antibodies which bind to a polypeptide comprising an amino acid sequence encoded by an HCV alternate reading frame in the body fluid or cells of a subject, wherein the presence of the polypeptide or antibodies which bind to the polypeptide is indicative of liver cancer or advanced cirrhosis.

28. The method of claim 27, wherein the polypeptide is the polypeptide of claim 1.

29. A method of diagnosing liver cancer or advanced cirrhosis, comprising detecting the presence or absence of antibodies in the body fluid of a subject, wherein the antibodies react with a polypeptide comprising an amino acid sequence encoded by an HCV alternate reading frame or a consensus polypeptide comprising a consensus sequence based on HCV alternate reading frame proteins encoded by more than one viral genotype, and wherein the presence of the antibodies is indicative of liver cancer or advanced cirrhosis.

30. The method of claim 29, wherein the polypeptide is the polypeptide of claim 1.

31. A method of diagnosing liver cancer or advanced cirrhosis, comprising detecting the presence or absence of a polypeptide in the body fluid or cells of a subject, wherein the polypeptide comprises an amino acid sequence encoded by an HCV alternate reading frame, and wherein the presence of the polypeptide is indicative of liver cancer or advanced cirrhosis.

32. The method of claim 31, wherein the polypeptide is the polypeptide of claim 1.

33. The method of claim 25 or 31, wherein the presence or absence of the polypeptide is detected by contacting a test sample comprising body fluid or cells of a subject with an antibody under conditions which allow the binding of the polypeptide to the antibody and wherein the binding of the antibody to the polypeptide in the sample indicates the presence of an HCV alternate reading frame polypeptide.

34. The method of claim 23 or 29, wherein the presence or absence of antibodies is detected by contacting a test sample comprising body fluid of a subject with the polypeptide under conditions which allow the binding of the polypeptide to the antibody and wherein the binding of the polypeptide to the antibody in the sample indicates the presence of an HCV alternate reading frame polypeptide.

35. The method of claim 34, wherein the polypeptide comprises an amino acid sequence that differs in sequence from a naturally occurring HCV alternate reading frame polypeptide by at least one amino acid residue.

36. The method of claim 35, wherein the entire polypeptide corresponds to a polypeptide encoded by a reading frame +1 or +2 to the standard hepatitis C reading frame.

37. The method of claim 35, wherein the polypeptide comprises an amino acid sequence at least about 80% identical to the consensus amino acid sequence shown in FIG. 9 using FASTA alignment.

38. The method of claim 37, wherein the polypeptide comprises an amino acid sequence at least about 90% identical to the consensus amino acid sequence shown in FIG. 9 using FASTA alignment.

39. The method of claim 38, wherein the polypeptide comprises an amino acid sequence at least about 95% identical to the consensus amino acid sequence shown in FIG. 9 using FASTA alignment.

40. The method of claim 33, wherein the antibody is polyclonal.

41. The method of claim 33, wherein the antibody is monoclonal.

42. An antibody that recognizes the polypeptide of claim 1.

43. A kit for detection of HCV or for detecting a particular clinical state comprising the antibody of claim 41.

44. A kit for detection of HCV or for detecting a particular clinical state comprising the polypeptide of claim 1.

45. A method for determining the presence or absence of antibodies that recognize the polypeptide of claim 1 in a test sample, comprising:
i) adsorbing at least one polypeptide of claim 1 to a matrix;
ii) contacting the test sample with the polypeptide(s) under conditions which allow the binding of antibodies present in the test sample to the polypeptide;
iii) removing the unbound sample from the matrix; and
iv) detecting the presence or absence of antibodies bound to the polypeptide(s) to thereby determine the presence or absence of antibodies that recognize the polypeptide of claim 1 in a test sample

46. A method for identifying a compound which interacts with the polypeptide of claim 1 or the nucleic acid molecule encoding the polypeptide, comprising:
contacting said polypeptide with a compound in a cell-free system under conditions which allow interaction of the compound with the polypeptide or the nucleic acid molecule encoding the polypeptide such that a complex is formed;
separating the compounds which do not form complexes with the polypeptide or the nucleic acid molecule encoding the polypeptide from those which do form complexes with the polypeptide or the nucleic acid molecule encoding the polypeptide; and
isolating and identifying the compounds which form complexes with the polypeptide or the nucleic acid molecule encoding the polypeptide to thereby identify a compound which interacts with the polypeptide of claim 1 or the nucleic acid molecule encoding the polypeptide.

47. A compound detected by the method of claim 46.
